(19) United States
(12) Patent Application Publication
(10) Pub. No.: US 2003/0176456 A1
(43) Pub. Date: Sep. 18, 2003

(54) Methods for reducing alcohol cravings in chronic alcoholics

(76) Inventors: Harry L. June, Indianapolis, IN (US); James M. Cook, Whitefish Bay, WI (US); Chunrong Ma, San Diego, CA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/329,100

(22) Filed: Dec. 23, 2002

Related U.S. Application Data
(60) Provisional application No. 60/345,417, filed on Dec. 21, 2001.

(51) Int. Cl. A61K 31/4745

(52) U.S. Cl. 514/292

(57) ABSTRACT

Methods are provided to reduce the anxiety associated with alcohol withdrawal in chronic alcoholics.

### CROSS-REFERENCE TO RELATED APPLICATION

This application claims priority of U.S. provisional application serial No. 60/345,417, filed Dec. 21, 2001.

Pursuant to 35 U.S.C. §202(c), it is hereby acknowledged that the U.S. Government has certain rights in the invention described herein, which was made in part with funds from the National Institutes of Alcohol Abuse and Alcoholism (NIAAA), grant Nos: AA10406 and AA11555, and National Institute of Mental Health (NIMH), grant No.: MH 46851.

### FIELD OF THE INVENTION

This invention relates to methods for the treatment of alcoholism. More specifically, the invention provides methods for reducing the anxiety associated with alcohol withdrawal.

### BACKGROUND OF THE INVENTION

Various scientific articles and patents are cited throughout the specification. Full citations for the references and patents are found within the specification and are incorporated by reference herein to describe the state of the art to which this invention pertains.

Alcohol addiction and dependence remain a significant public health concern, impacting physical and mental well-being, family structure and occupational stability (Kessler et al., 1997). While advances have been made in the development of novel therapies to treat alcoholism (O'Malley et al., 1992; Volpicelli et al. 1992; Kranzler, 2000; Spanagel and Zieglgansberger, 1997), alcohol-dependent individuals represent a heterogeneous group (Cloninger, 1987; Li et al. 1991; 2000), and it is unlikely that a single pharmacological treatment will be effective for all alcoholics. Hence, a better understanding of the neuromechanisms which regulate alcohol seeking behaviors and the design of clinically safe and effective drugs that reduce alcohol addiction and dependence remain a high priority (Kranzler, 2000; Johnson and Daoud, 2000). While the precise neuromechanisms regulating alcohol-seeking behaviors remain unknown, there is now compelling evidence that the GABA_{A} receptors within the striatopallidal and extended amygdala system are involved in the “acute” reinforcing actions of alcohol (Koob, 1998; Koob et al., 1998; June et al., 1998c; McBride and Li, 1998). The striatopallidal and extended amygdala system include the sublenticular extended amygdala [substantia innominata-ventral pallidum (VP)], shell of the nucleus accumbens, and central nucleus of the amygdala (Heimer et al., 1991; Heimer and Alheid, 1991). Among the potential GABA_{A} receptor isoforms within the VP regulating alcohol-seeking behaviors, GABA_{A} receptors containing the α1 receptor subtype (GABA_{A1}) appear preeminent. Thus, Criswell et al., (1993, 1995) observed that acute alcohol administration selectively enhanced the effects of ionotophoretically applied GABA in the VP. However, no effects were seen in the septum, VTA, and CAI hippocampus. Further, a positive correlation was observed between alcohol-induced GABA enhancement and [³H] zolpidem binding (an α1 subtype selective agonist). Other investigators have identified a dense reciprocal projection from the VP to the NACC (Nauta et al., 1978; Zahm and Heimer, 1988; Groenewegen et al., 1993), and many of these have been found to be GABAergic neurons (Mogenson and Nielson, 1983; Kuo and Chang, 1992; Churchill and Kalivas, 1994). The NACC is well established as a substrate that regulates the reinforcing properties of abused drugs (Koob, 1998; Koob et al., 1998). Finally, immunohistochemical (Turner et al., 1993; Fritschy and Mohler, 1995) and in situ hybridization studies (Churchill et al., 1991; Wisden et al., 1992; Duncan et al., 1995) have demonstrated that the VP contains one of the highest concentrations of mRNA encoding the α1 subunit in the CNS. These findings, together with pharmacological studies suggesting the VP plays a role in reward-mediated behaviors of psychostimulants and opiates (Hubner and Koob, 1990; Napier and Chrobak, 1992; Churchill and Kalivas, 1994; Gong et al., 1996; 1997), suggest a possible role of the VP-α1 receptors in the euphoric properties of alcohol.

### SUMMARY OF THE INVENTION

In accordance with the present invention, methods are provided for reducing the anxiety associated with alcohol withdrawal in chronic alcoholics or other patients in need of treatment for anxiety. Exemplary methods include the administration of an antagonist of α1 containing GABA_{A} receptors in an amount effective to reduce anxiety in the patient. Such antagonists of α1 containing GABA_{A} receptors include, but are not limited to, βCCt and 3-PBC. The antagonists of the invention may be administered separately or together to reduce anxiety in the chronic alchoholic. Optionally, naltrexone may also be administered with the antagonists of the invention.

In a preferred embodiment, the anti-anxiolytic agents of the invention are administered in the range of about 20 to about 60 mg with 40 mg being most preferred. It is also preferred that the agents be administered about 3-4 times a week.

### BRIEF DESCRIPTION OF THE DRAWINGS

FIG. 1. Synthesis and structure of 3-PBC (3-Propoxy-β-carboline hydrochloride), the α1 selective benzodiazepine antagonist.

FIG. 2. Modulation of GABA_{A} α₁β_{3γ2}, α₂β_{3γ2}, α₃β_{3γ2}, α₄β_{3γ2}, and α₅β_{3γ2} receptor subunit combinations expressed in Xenopus oocytes by 3-PBC (open bars), Ro 15-1788 (flumazenil) (hatched bar) and ZK 93426 (black bars). A saturating concentration of modulator (1-10 μM) was co-applied over voltage clamped oocytes along with an EC₅₀ of GABA. The whole cell current response in the presence of modulator is reported as a percentage of the current response to GABA alone (% GABA response). Each value is the mean±S.D. of at least 3 separate oocytes.

FIG. 3A-I. Actions of 3-PBC, flumazenil and ZK 93426 on recombinant GABA_{A} receptor subtypes. Top, current responses of voltage-clamped oocytes expressing GABA_{A} α1β3γ2 receptors A, during application of 50 μM (EC₅₀) GABA alone for duration indicated by black bar (left trace). Current response from the same oocyte subsequently co-applied with 50 μM GABA along with 10 μM 3-PBC for duration indicated by open bar (right trace). B, current response of a voltage-clamped oocyte during application of 50 μM GABA for duration indicated by black bar (left trace). Current response from same oocyte subsequently co-applied with 50 μM GABA along with 1 μM Flumazenil for duration indicated by open bar (right trace). C, current response of a voltage-clamped oocyte during application of 50 μM GABA for duration indicated by black bar (left trace). Current response from same oocyte subsequently co-applied with 50 μM GABA along with 10 μM ZK-93426 for duration indicated by open bar (right trace). Center, current responses of voltage-clamped oocytes expressing GABA_{A} α2β3γ2 receptors D, during application of 50 μM (EC₅₀) GABA for duration indicated by black bar (left trace). Current response from same oocyte subseqently co-applied with 50 μM GABA along with 10 μM 3-PBC for duration indicated by open bar (right trace). E, current response of a voltage-clamped oocyte during application of 50 μM GABA for duration indicated by black bar (left trace). Current response from same oocyte subsequently co-applied with 50 μM GABA along with 10 μM Flumazenil for duration indicated by open bar (right trace). F, current response of a voltage-clamped oocyte during application of 50 μM GABA for duration indicated by black bar (left trace). Current response from same oocyte subsequently co-applied with 50 μM GABA along with 10 μM ZK-93426 for duration indicated by open bar (right trace). Bottom, current responses of voltage-clamped oocytes expressing GABA_{A} α3β3γ2 receptors G, during application of 30 μM (EC₅₀) GABA for duration indicated by black bar (left trace). Current response from same oocyte subseqently co-applied with 30 μM GABA along with 10 μM 3-PBC for duration indicated by open bar (right trace). H, current response of a voltage-clamped oocyte during application of 30 μM GABA for duration indicated by black bar (left trace). Current response from same oocyte subsequently co-applied with 30 μM GABA along with 1 μM Flumazenil for duration indicated by open bar (right trace). I, current response of a voltage-clamped oocyte during application of 30 μM GABA for duration indicated by black bar (left trace). Current response from same oocyte subsequently co-applied with 30 μM GABA along with 10 μM ZK-93426 for duration indicated by open bar (right trace). Scale bars: 5 nA, 10 s.

FIG. 4. Dose-response of systemic (0.0-20 mg/kg) (N=13) [A], and bilateral infusions of 3-PBC (0.5-40 μg) in the VP (N=12) [B] and NACC/caudate putamen (neuroanatomical control loci) (N=7) [C] on a concurrent fixed-ratio (FR4) schedule for EtOH (10% v/v) and saccharin (0.025% or 0.05% w/v) responding during the 1 hr operant session. *P≦0.05 vs the control conditions values by ANOVA and post hoc Newman-Keuls test. Bars represent±S.E.M. in this and subsequent figures. The two control conditions were pooled in the systemic group and compared against the drug treatment conditions [see results section].

FIG. 5. Reconstruction of serial coronal sections of the rat brain illustrating the bilateral guide cannula tips for the ventral pallidum (VP) [anterior to medial division] (N=12) [A], NACC and caudate putamen (N=7) rats (i.e., neuroanatomical controls) [B] included in the data depicted in FIGS. 4A and 4B, respectively. Each rat is represented by two solid black circles: one in the left, and one in the right hemisphere. Coronal sections are adapted from the rat brain atlas of Paxinos and Watson, 1998, reproduced with permission from Academic Press.

FIG. 6. Representative histological photomicrographs for four rats illustrating coronal sections of the VP [anterior to medial division] [A-D]. The photomicrographs depict the guide cannulae tracks and the magnitude of cellular damage caused by the bilateral cannula implantation.

FIG. 7. Representative histological photomicrographs for three rats illustrating coronal sections for two NACC [A-B] and one caudate putamen [C] rat. The photomicrographs depict the guide cannulae tracks and the magnitude of cellular damage caused by the bilateral cannula implantation.

FIG. 8. Cumulative time course profiles across the 60 min interval for EtOH [A] and saccharin-maintained [B] responding relative to the pooled control condition following systemic injections of 3-PBC. The data are redrawn from FIG. 4A. All 3-PBC doses suppressed the initiation of EtOH responding during the first 10 and 20 min intervals (p≦0.05) [see results section]. In contrast to EtOH responding, except for the 20 mg/kg dose, beginning at the 30 min interval, and throughout the remainder of the 60 min session, all PBC doses significantly elevated saccharin-maintained responding (p≦0.05).

FIG. 9. Cumulative time course profiles across the 60 min interval for EtOH [A] and saccharin-maintained [B] responding relative to the pooled control condition following infusions of 3-PBC in the VP. The data are redrawn from FIG. 4B. All 3-PBC infusions suppressed the initiation of EtOH responding during the first 10 and 20 min intervals (p≦0.05). Except for the 0.5 μg dose condition, all 3-PBC infusions continued to significantly suppressed responding throughout the 30-60 intervals (p≦0.05). In contrast to EtOH maintained responding, except for the 40 μg dose condition (p≦0.05), none of the 3-PBC infusions altered responding maintained by saccharin at the 10 min interval (p>0.05). Similar to its effect on EtOH-maintained responding, the suppression with the 40 μg dose was sustained throughout the remainder of the 60 min session (p≦0.05).

FIG. 10. Synthesis and structure of βCCt.

FIG. 11. Modulation of GABA_{A} α₁β_{3γ2}, α₂β_{3γ2}, α₂β_{3γ2}, α₄β_{3γ2}, and α₅β_{3γ2} receptor subunit combinations expressed in Ltk cells by βCCt (open bars), flumazenil (shaded bars), and ZK 93426 (black bars). A saturating concentration (1-10 μM) was co-applied over voltage clamped oocytes along with an EC₅₀ of GABA. Each value is the mean % GABA response±S.D. of at least 4 separate oocytes.

FIG. 12. Actions of βCCt, flumazenil and ZK 93426 on recombinant GABA_{A} receptor subtypes. Top, current responses of voltage-clamped oocytes expressing GABA_{A} α1β3 γ2 receptors (a), during application of 50 μM (EC₅₀) GABA alone for duration indicated by black bar (left trace). Current response from the same oocyte subsequently co-applied with 50 μM GABA along with 10 μM βCCt for duration indicated by open bar (right trace). (b), current response of a voltage-clamped oocyte during application of 50 μM GABA for duration indicated by black bar (left trace). Current response from same oocyte subsequently co-applied with 50 μM GABA along with 1 μM flumazenil for duration indicated by open bar (right trace). (c), current response of a voltage-clamped oocyte during application of 50 μM GABA for duration indicated by black bar (left trace). Current response from same oocyte subsequently co-applied with 50 μM GABA along with 10 μM ZK-93426 for duration indicated by open bar (right trace). Center, current responses of voltage-clamped oocytes expressing GABA_{A} α2β3γ2 receptors (d), during application of 50 μM (EC₅₀) GABA for duration indicated by black bar (left trace). Current response from same oocyte subsequently co-applied with 50 μM GABA along with 10 μM βCCT for duration indicated by open bar (right trace). (e), current response of a voltage-clamped oocyte during application of 50 μM GABA for duration indicated by black bar (left trace). Current response from same oocyte subsequently co-applied with 50 μM GABA along with 10 μM flumazenil for duration indicated by open bar (right trace). (f), current response of a voltage-clamped oocyte during application of 50 μM GABA for duration indicated by black bar (left trace). Current response from same oocyte subsequently co-applied with 50 μM GABA along with 10 μM ZK-93426 for duration indicated by open bar (right trace). Bottom, current responses of voltage-clamped oocytes expressing GABA_{A} α3β3γ2 receptors (g), during application of 30 μM (EC₅₀) GABA for duration indicated by black bar (left trace). Current response from same oocyte subsequently co-applied with 30 μM GABA along with 10 μM βCCt for duration indicated by open bar (right trace). (h), current response of a voltage-clamped oocyte during application of 30 μM GABA for duration indicated by black bar (left trace). Current response from same oocyte subsequently co-applied with 30 μM GABA along with 1 μM flumazenil for duration indicated by open bar (right trace). (i), current response of a voltage-clamped oocyte during application of 30 μM GABA for duration indicated by black bar (left trace). Current response from same oocyte subsequently co-applied with 30 μM GABA along with 10 μM ZK-93426 for duration indicated by open bar (right trace). Scale bars: 5 nA, 10 s.

FIG. 13. Dose-response of systemic βCCt injections (i.p.) in (a) P (Ps) (5-40 mg/kg) and (b) HAD-1 (Hads) (1-10 mg/kg) rats. P rats (N=11) performed under a concurrent fixed-ratio (FR4) schedule for EtOH (10% v/v) and saccharin (0.05% w/v). HAD-1 rats (N=11) performed under an alternate-day access paradigm wherein they received EtOH (10% v/v) on day 1, and sucrose (1% w/v) on day 2. Fifteen min after the i.p. injections rats were placed in the operant chamber to lever press for a 60 min session. **p<0.01, *p<0.05 vs the control conditions values by ANOVA and post hoc Newman-Keuls test. Bars represent±S.E.M. in this and subsequent figures.

FIG. 14. Reconstruction of serial coronal sections of the “P” rat brains illustrating the bilateral guide cannula tips for the (a) ventral pallidum (VP) [anterior to posterior division] (N=11) and (b) nucleus accumbens (NACC)/caudate putamen (Cpu) (N=7) (i.e., neuroanatomical controls). Each rat is represented by two solid black circles: one in the left, and one in the right hemisphere. Coronal sections are adapted from the rat brain atlas of Paxinos and Watson, 1998, reproduced with permission from Academic Press.

FIG. 15. Representative histological photomicrographs of bilaterally implanted cannulas in four “P rats” terminating in the (a) anterior (Bregma+0.70 mm), (b) subcommissural (Bregma +0.20 mm), (c) medial VP (Bregma −0.26 mm), and (d) posterior VP (Bregma −0.80 mm). The photomicrographs depict the distal ends of the cannula tracks.

FIG. 16. (a) Performance of female P rats (n=1) on a concurrent fixed-ratio (FR-4) schedule for EtOH (10% v/v) and saccharin (0.05% w/v) following bilateral infusions of βCCt (0.0-40 μg) in the VP. (b) Performance of control female P rats (n=7) on a concurrent FR-4 schedule for EtOH (10% v/v) and saccharin (0.05% w/v) following bilateral infusions of βCCt (0.0-40 μg) in the NACC/CPu areas. (c, d) Performance of female Had rats (n=9) on an FR-4 schedule for EtOH (10% v/v) following unilateral infusions of βCCt (0.0-7.5 μg) in the VP on the first day of infusion and 24 hr post-drug administration. (e) Performance of the same female Had rats in FIG. c (n=9) on an FR-4 schedule for EtOH (10% v/v) following unilateral infusions of βCCt (0.0-7.5 μg) in the NACC/CPu areas on the first day of infusion. **p≦0.01, *p≦0.05, compared with the baseline (BT) and artificial cerebral spinal fluid (aCSF) conditions using post-hoc Newman Keuls Tests.

FIG. 17. Reconstruction of serial coronal sections of the HAD-1 rat brains illustrating the unilateral guide cannula tips for the (a) NACC/CPu (n=9) (i.e., neuroanatomical controls) and (b) VP [anterior to posterior division] (n=9). Each rat is represented by two solid black circles: one in the left NACC/CPu and one in the right VP [Total N=9]. Coronal sections are adapted from the rat brain atlas of Paxinos and Watson, 1998, reproduced with permission from Academic Press.

FIG. 18. Representative histological photomicrographs of two “HAD-1 rats” with one unilateral cannula terminating in the (a, c) NACC/CPu (i.e., neuroanatomical control loci) and the second unilateral cannula terminating in the (b, d) anterior (Bregma +0.70 mm) to medial VP (Bregma −0.26 mm).

FIG. 19. Representative histological photomicrographs of two additional “HAD-1 rats” with one unilateral cannula terminating in the (a, c) NACC/CPu (i.e., neuroanatomical control loci) and the second unilateral cannula terminating in the (b, d) medial (Bregma −0.26 mm), to posterior VP (Bregma −0.80 mm).

FIG. 20. Evaluation of βCCt's capacity to antagonize the locomotor sedation produced by chlordiazepoxide (CZ) in (a) HAD-1 rats in the vehicle [n=9], 10 mg/kg CZ [n=6], 15 mg/kg βCCt+10 mg/kg CZ [n=7], 1.25 g/kg EtOH [n=7], 15 mg/kg βCCt+1.25 g/kg EtOH [n=7], and 15 mg/kg βCCt [n=6] treatment groups and (b) P rats in the vehicle [n=8], 10 mg/kg CZ [n=6], 15 mg/kg βCCt+10 mg/kg CZ [n=7], 1.25 g/kg EtOH [n=7], 15 mg/kg βCCt+1.25 g/kg EtOH [n=7], and 15 mg/kg βCCt [n=7] treatment groups. Data are ambulatory count in an open field (mean±s.e.m.) for 10 min. ** p≦0.01, compared with the vehicle (Veh) using post-hoc Newman Keuls Tests. Plus sign, p<0.01 compared with the 1.25 g/kg EtOH condition. βCCt only partially antagonized the EtOH sedation in P rats.

FIG. 21. [A] Agonist and inverse-agonist activity is seen at the α1 receptor in HEK cells following 0.1 to 100 μM βCCt. βCCt appears to modulate GABA induced currents via the benzodiazepine binding-site in HEK cell following 0.1 to 100 μM βCCt. [B] Intrinsic activity is seen in HEK cells following 0.1 to 100 μM βCCt.

FIG. 22. [A] Strong agonist activity is seen at the α2 receptor in HEK cells following 0.1 to 100 μM βCCt. [B] Intrinsic activity is seen in HEK cells following 0.1 to 100 μM βCCt.

FIG. 23. [A] Strong agonist activity is seen at the α3 receptor in HEK cells following 0.1 to 100 μM βCCt. [B] Intrinsic activity was not tested.

FIG. 24. [A] Moderate agonist activity is seen at the α4 receptor in HEK cells following 0.1 to 100 μM βCCt. βCCt appears to modulate GABA induced currents via the benzodiazepine binding-site in HEK cell following 0.1 to 100 μM βCCt. [B] Intrinsic activity is seen in HEK cells following 0.1 to 100 μM βCCt.

FIG. 25. [A] Agonist and inverse-agonist activity is seen at the α5 receptor in HEK cells following 0.1 to 100 μM βCCt. [B] Intrinsic activity is seen in HEK cells following 0.1 to 100 μM βCCt.

FIG. 26. [A] Minimal agonist activity is seen at the α6 receptor in HEK cells following 0.1 to 100 μM βCCt. βCCt appears to modulate GABA induced currents via the benzodiazepine binding-site in HEK cell following 0.1 to 100 μM βCCt. [B] Intrinsic activity is seen in HEK cells following 0.1 to 100 μM βCCt.

FIG. 27. βCCt (5-30 mg/kg) produces anti-anxiety effects in HAD rats [A & C]. βCCt (5-60 mg/kg) produces anti-anxiety effects in P rats [B & D].

FIG. 28. Comparison of βCCt (5 & 15 mg/kg) with chlordiazepoxide (CZ) (2.5 & 5 mg/kg) in HAD [A] and P [B] rats on an elevated plus maze. βCCt is as equally effective as chlordiazepoxide in reducing anxiety. βCCt fails to antagonize the anti-anxiety effects of chlordiazepoxide. Comparison of 10 mg/kg of chlordiazepoxide (CZ) and EtOH (1.25 mg/kg) as sedative agents in the open field measured as activity counts. βCCt blocks the sedation produced by both chlordiazepoxide and an intoxicating dose of EtOH in HAD [C] and P [D] rats. Given alone, βCCt does not produce any effects on motor activity.

FIG. 29. [A] βCCt (15 & 30 mg/kg) significantly reduces EtOH responding in P rats following oral (gavage) administration. [B] 24 hours after oral (gavage) administration selected doses (15 & 75 mg/kg) of βCCt significantly reduce EtOH responding. [C] βCCt (75 mg/kg) fails to alter responding for sucrose.

FIG. 30. [A] 3-PBC (30-75 mg/kg) dose-dependently reduces EtOH responding in P rats following oral (gavage) administration. [B] 3-PBC (75 mg/kg) fails to alter responding for sucrose.

FIG. 31. [A] Naltrexone® (30-75 mg/kg) significantly reduces EtOH responding in P rats following oral (gavage) administration. [B] Naltrexone® (75 mg/kg) significantly reduces responding for sucrose.

### DETAILED DESCRIPTION OF THE INVENTION

Important advances have been made in the development of new drugs to treat alcoholism. However, alcohol-dependent individuals represent a heterogeneous group and thus a variety of pharmacological regimens are required for the effective treatment of all alcoholics. The design of clinically safe and effective drugs that reduce alcohol addiction and dependence remains a high priority in the field of alcoholism research. The current drugs on the market (e.g., opiate antagonists) exhibit certain unwanted side effects rendering them useless in many patients. The present invention provides methods for reducing alcohol drinking behavior in humans. The invention also encompasses methods for reducing the anxiety that chronic alcoholics experience upon withdrawal from alcohol. The agents utilized in the methods of the invention are antagonists of the α₁ subtype GABA_{A} receptor, such as, βCCt and 3-PBC, which do not appear to generate the unwanted side effects observed with the opiate antagonists.

3-PBC

The reinforcing actions of alcohol have been linked to GABA_{A} receptors within the CNS; however, the heterogeneity in traditional alcohol reward substrates precludes study of the precise GABA_{A} receptor subtype. To explore the role of the α₁ receptor, we developed a selective benzodiazepine (BDZ) site ligand, 3-PBC, which binds to the α₁-subtype of the GABA_{A} receptor. Compared with the prototype BDZ I agonist, zolpidem, 3-PBC exhibits a slightly higher binding selectivity for the α₁ receptor (Cox et al., 1998; Huang et al., 2000) (Table 1) and thus provides a superior agent for the treatment of alcoholics. While the α₁ receptor is expressed extensively in several CNS loci [e.g., cortex, ventral pallidum (VP) cerebellum], the VP has been shown to be an important neurobiological substrate in the rewarding actions of psychostimulants and opioids. In the present study, following bilateral microinfusion of 3-PBC (0.5-40 μg), we evaluated the functional significance of the VP-α₁ receptors in regulating the rewarding properties of alcohol. Our results demonstrated that activation of the α₁ receptors in the anterior and medial VP produced marked reductions on alcohol-maintained responding in a genetically selected rodent model of alcohol drinking. The VP infusions showed both neuroanatomical and reinforcer specificity, as no effects were seen in sites dorsal to the VP (e.g., nucleus accumbens, caudate putamen). Saccharin-maintained responding was reduced only with the highest dose (e.g., 40 μg). 3-PBC's inability to produce adverse side effects in vivo paralleled its weak partial agonist profile at recombinant diazepam-sensitive receptors (e.g., α1β3γ2, α2β3γ2 and α3β3γ2) in vitro. Together, these results demonstrate that the α₁ containing GABA_{A} receptors in both the anterior and medial VP are important in regulating the euphoric properties of alcohol. Thus, agents with binding affinity for these receptors, such as, 3-PBC, represent ideal pharmacological agents for the treatment of alcohol-dependent subjects.

TABLE 1
Binding affinities at recombinant receptors (αₓβ₃γ₂) for
prototypical competitive BDZ antagonists (top panel) and the currently
known α₁ subunit selective ligands (bottom panel), Ki Values in nM^{a}

| Compound | α₁ | α₂ | α₃ | α₅ | α₆ |
|---|---|---|---|---|---|
| ZK 93426^{b} | 11 | 31 | 24 | 3 | 1600 |
| Ro15-1788 | 0.8 | 0.9 | 1.05 | 0.6 | 148 |
| 3-PBC | 5.3 | 52.3 | 68.8 | 591 | >1000 |
| 3-EBC | 6.43 | 25.1 | ND | 868 | >1000 |
| βCCt | 0.72 | 15 | 18.9 | 111 | >1000 |
| Zolpidem | 26.7 | 156 | 383 | >1000 | >1000 |
| CL218, 872 | 57 | 1964 | 1161 | 561 | >1000 |
| L-838, 417^{c} | 0.79 | 0.67 | 0.67 | 267 | 2.25 |

^{a}Cox et al., 1995;
^{b}Pribilla et al., unpublished from Shering Labs;
^{c}McKernan et al., 2000

βCCt

In the present study, we also developed βCCt, a mixed agonist-antagonist benzodiazepine (BDZ) site ligand with binding selectivity at the α₁ subtype GABA_{A} receptor. The in vivo actions of βCCt were then determined following microinfusion into the ventral pallidum (VP), a novel alcohol reward substrate, which primarily expresses the α₁ receptor. In two selectively-bred rodent models of chronic alcohol drinking [e.g., HAD-1, P rats], bilateral microinfusion of βCCt (0.5-40 μg) produced marked reductions in alcohol-reinforced behaviors. Further, VP infusions of βCCt exhibited both neuroanatomical and reinforcer specificity. Thus, no effects on alcohol-reinforced behaviors were observed following infusion in the nucleus accumbens (NACC)/caudate putamen (CPu), or on responding maintained by saccharin. Parenternal administered βCCt (1-40 mg/kg) was equally effective and selective in reducing alcohol-reinforced behaviors in P and HAD-1 rats. Additional tests of locomotor activity revealed βCCt reversed the locomotor sedation produced by both chlordiazepoxide (10 mg/kg) and EtOH (1.25 g/kg), but was devoid of intrinsic effects given alone. Studies in recombinant receptors expressed in Xenopus oocytes revealed βCCt acted as a low efficacy partial agonist at α3β3γ2 and α4β3γ2 receptors and as a low efficacy inverse agonist at α1β3γ2, α2β3γ2 and α5β3γ2 receptors. The present studies indicate that βCCt is capable of antagonizing the reinforcing and the sedative properties of alcohol. These anti-alcohol properties of βCCt are primarily mediated via the α₁ subtype GABA_{A} receptor. βCCt, thus, may be used as a pharmacotherapeutic agent to effectively reduce alcohol drinking behavior in human alcoholics.

Anti-anxiety Property

The present invention provides that 3-PBC and βCCt display selective alcohol suppressant effects. It is also demonstrated that βCCt displays agonist effects to α₂ subtype GABA_{A} receptors, whereas the α₂ subtype GABA_{A} receptors are involved in regulating anxiety. Moreover, it is evidenced that βCCt possess anti-anxiety effects like chlordiazepoxide and that βCCt exhibits anti-sedation effects in blocking chlordiazepoxide and intoxicating dose of alcohol.

Pharmaceutical Composition and Administration

The compound of this invention, an antagonist of α1 containing GABA_{A} receptors, e.g. βCCt and 3-PBC, will be administered in a therapeutically effective amount by any of the accepted modes of administration for agents that serve similar utilities. The compounds of the invention may be administered separately or combined with each other or other agents known to be effective for the treatment of alcoholism (e.g., naltrexone). The actual amount of the compound of this invention, i.e., the active ingredient, will depend upon numerous factors such as the severity of the disease to be treated, the age and relative health of the subject, the potency of the compound used, the route and form of administration, and other factors.

Therapeutically effective amounts of the compounds may range from approximately 0.1-50 mg per kilogram body weight of the recipient per day; preferably about 0.5-20 mg/kg/day. Thus, for administration to a 70 kg person, the dosage range would most preferably be about 40 mg to 1.4 g per day.

The compositions of the invention may be prepared in various forms for administration, including tablets, caplets, pills, or dragees, or can be filled in suitable containers, such as capsules, or, in the case of suspensions, filled into bottles. As used herein, “pharmaceutically acceptable carrier medium” includes any and all solvents, diluents, other liquid vehicle, dispersion or suspension aids, surface active ingredients, preservatives, solid binders, lubricants, and the like, as suited to the particular dosage form desired. Remington's Pharmaceutical Sciences, Fifteenth Edition, E. W. Martin (Mack Publishing Co., Easton Pa. 1975) discloses various vehicles or carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of the invention.

In the pharmaceutical combination compositions of the invention, the active agents may be present in an amount of at least about 0.1% and not more than about 95% by weight, based on the total weight of the compositions, including carrier medium and auxiliary agent(s). Preferably, the proportion of active agent varies between about 1% and about 75% by weight of the composition. Pharmaceutical organic or inorganic solid or liquid carrier media suitable for enteral or parenteral administration can be used to make up the composition. Gelatine, lactose, starch, magnesium, stearate, talc, vegetable and animal fats and oils, gum, polyalkylene glycol, or other known excipients or diluents for medicaments may all be suitable as carrier media.

The compositions described herein are preferably formulated in dosage unit form for ease of administration and uniformity of dosage. A “dosage unit form” as used herein refers to a physically discrete unit of pharmaceutical composition for the patient to be treated. Each dosage should contain the quantity of active material calculated to produce the desired therapeutic effect either as such, or in association with the selected pharmaceutical carrier medium. The pharmaceutical compositions of the invention may be administered orally, parenterally, by intramuscular injection, intraperitoneal injection, intravenous infusion, or the like. Intravenous administration is particularly preferred.

The following examples are provided to facilitate the practice of the present invention. They are not intended to limit the invention in anyway.

### EXAMPLE I

### Selective Reduction of Alcohol Responding by 3-PBC

Material and Methods

Subjects

Male selectively-bred alcohol-preferring (P) rats (N=33) from the S48 generation (Lumeng et al. 1995) were obtained from the Alcohol Research Center at Indiana University School of Medicine. All animals were approximately 3-4 months of age and weighed between 261 and 381 g at the beginning of the experiment. Animals were individually housed in wire-mesh stainless steel cages or plastic tubs. The vivarium was maintained at an ambient temperature of 21° C. and was on a normal 12 hr light/dark cycle. Rats were provided ad libitum access to food and water, except during the first 2 days of the training phase wherein rats were fluid deprived 23 hr daily (see below). Thereafter, rats were maintained on ad libitum food and water. All training and experimental sessions took place between 9 a.m. to 4 p.m. The treatment of all subjects was approved by the institutional review board within the School of Science at IUPUI. All procedures were conducted in strict adherence with the NIH Guide for the Care and Use of Laboratory Animals.

Drug and Solutions

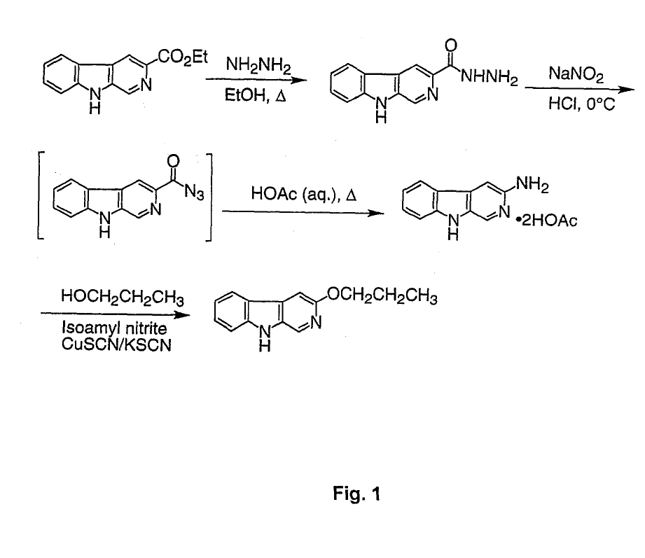

3-PBC (3-Propoxy-β-carboline hydrochloride) was synthesized via modification of the prototypical inverse agonist, βCCE as outlined previously (Cox et al., 1998). The structure of 3-PBC is shown in FIG. 1. For systemic drug administrations, 3-PBC was prepared as an emulsion in a Tween-20 (Sigma Chemical Co., St. Louis, Mo.) solution which comprised 99.80 mls of a 0.90% sodium chloride solution and 0.20 mls of Tween-20. All drug solutions were mildly sonicated (Fisher Scientific, Springfield, N.J.) to aid in dissolving the compound. The Tween-20 vehicle solution was administered as the control injection for the systemic experiment. Systemic injections were given intraperitoneally (i.p) in an injection volume of 1 ml/kg. For the microinjection studies, 3-PBC was dissolved in artificial cerebrospinal fluid (aCSF) (see below).

Radioligand Binding

[³H] Diazepam binding to rat cerebral cortical membranes was accomplished by using a modification of the method previously described (Cox et al., 1998). In brief, rats were sacrificed by decapitation, and the cerebral cortex removed. Tissue was disrupted in 100 volumes of Tris-HCl buffer (50 mM, pH 7.4) with Polytron (15 s setting 6-7, Brinkman Instruments, Westbury, N.Y) and centrifuged (4° C.) for 20 min at 2000 g. Tissue was resuspended in an equal volume of buffer and recentrifuged. This procedure was repeated a total of three times and the tissue resuspended in 50 volumes of buffer. Incubations (1 mL) consisted of tissue (0.3 mL), drug solution (0.1 mL), buffer (0.5 mL) and radioligand (0.1 mL). Incubations (4° C.) were initiated by addition of [³H] diazepam, (final concentration, 2 mM; specific activity, 76 Ci/mmol, Du Pont-NEN, Boston Mass.) and terminated after 120 min by rapid filtration through GF/B filters and washing with two 5 mL aliquots of ice-cold buffer with a Brandel M-24R filtering manifold. Nonspecific binding was determined by substitution of nonradioactive flunitrazepam (final concentration, 10 μM) for the drug solution and represented <10% of the total binding. Specific binding was defined as the difference in binding obtained in the presence and absence of 10 μM flunitrazepam. The IC₅₀ values were estimated using Hill plots.

Xenopus Oocyte Expressions Assay

Xenopus Laevis frogs were purchased from Xenopus-1 (Dexter, Mich.). Collagenase B was from Boehringer Mannheim (Indianapolis, Ind.). GABA was obtained from RBI (Natick, Mass.). All compounds were prepared as a 10 mM stock solution in EtOH and stored at −20° C. cDNA clones. The rat GABA_{A} receptor α1, α5 and γ2 subunit clones were gifts from H.Luddens (Department of Psychiatry, University of Mainz, Germany). The rat GABA_{A} receptor β3 subunit clone was a gift from L. Mahan (NINDS, NIH). Capped cRNA was synthesized from linearized template cDNA encoding the subunits using mMESSAGE mMACHINE kits (Ambion, Austin, Tex.). Oocytes were injected with the α, β and γ subunits in a 1:1:1 molar ratio as determined by UV absorbance. Mature X. laevis frogs were anesthetized by submersion in 0.1% 3-aminobenzoic acid ethyl ester, and oocytes were surgically removed. Follicle cells were removed by treatment with collagenase B for 2 hr. Each oocyte was injected with 50-100 ng of cRNA in 50 nl of water and incubated at 19° C. in modified Barth's saline (88 mM NaCl, 1 mM KCl, 2.4 mM NaHCO₃, 0.41 mM CaCl₂, 0.82 mM MgSO₄, 100 μg/ml gentamicin, and 15 mM HEPES, pH 7.6). Oocytes were recorded after 3 to 10 days post-injection. Oocytes were perfused at room temperature in a Warner Instruments oocyte recording chamber #RC-5/18 (Hamden, Conn.) with perfusion solution (115 mM NaCl, 1.8 mM CaC₂, 2.5 mM KCT, 10 mM HEPES, pH 7.2). Perfusion solution was gravity fed continuously at a rate of 15 ml/min (see Harvey et al., 1997). Compounds were diluted in perfusion solution and applied until after a peak current was reached. Current responses to GABA application were measured under two-electrode voltage clamp at a holding potential of −60 mV. Data was collected using a GeneClamp 500 amplifier and Axoscope software (Axon Instruments, Foster City, Calif.). GABA concentration-response curves for the GABA_{A} receptor subunit combinations were constructed by normalizing responses to a low concentration of GABA to minimize variability, then re-normalized to the maximal response for comparison. Concentration-response data were fitted to a four parameter logistic using GraphPad Prizm, and the EC₅₀ for each receptor subtype was determined. Peak whole cell current responses of a voltage-clamped oocyte to an EC₅₀ concentration of GABA in the presence of saturating (1-10 μM) concentrations of modulators are reported as a percentage of the peak response to GABA alone (“percent GABA response” or “% control”).

Behavioral Testing Apparatus and Training Procedures

Behavioral testing was conducted in 15 standard operant chambers (Coulbourn Instruments) equipped with two removable levers and two dipper fluid delivery systems enclosed in sound-attenuated cubicles as previously described (June et al., 1998a,b). A concurrent fixed-ratio schedule was employed to investigate the capacity of systemic and direct microinjections of 3-PBC in the CNS to modify EtOH and saccharin-maintained responding. The specific details of these procedures have recently been described (June et al., 1998; 1999; 2001). In brief, rats were initially trained to orally self-administer EtOH and water in daily 60 min sessions on a concurrent FRI schedule in a two-lever choice situation. After a period of stabilization on the FRI schedule, the response requirement was increased to a concurrent FR4 schedule and the water reinforcer was replaced with saccharin (0.025-0.05% w/v). The importance of alternative and concurrently presented reinforcers in examining the positive reinforcing properties of drugs of abuse has been discussed previously (Meisch and Lemaire, 1993; June et al., 2001; June in press).

Systemic Drug Treatment Procedures

3-PBC was administered 15 min before the operant session to allow for optimal absorption and CNS distribution. 3-PBC was tested at doses of 1-10 mg/kg. The duration of the operant sessions was 60 min, however, subjects were tested at 24 and 48 hour post drug administration to determine any residual drug effects. A minimum of 72-96 hours were allocated between drug treatments to permit animals to return to baseline levels. This period prevented confounding of drug treatments due to residual effects (for details see June et al., 1998b).

Surgery and Microinfusion Procedures

Guide cannulas were stereotaxically implanted bilaterally in the anterior (AP +0.48; ML ±1.6; DV −7.2, with 6° lateral angle) (n=8) and medial (AP −0.26; ML ±2.5; DV −7.0) (n=6) VP. The neuroanatomical control rats were bilaterally implanted in either the caudate putamen or NACC. The coordinates for the caudate putamen were AP +1.5; ML ±2.5; DV 4.2 (n=3). In the NACC group some rats were implanted in the shell [AP +1.4; ML +0.8; DV −6.0] (n=3) while others were implanted in the core [AP +1.4; ML ±1.7; DV −5.71] (n=3). All coordinates are given in millimeters relative to Bregma based on the Paxinos and Watson (1998) atlas. Subjects were given 7 days to recover from surgery before returning to training in the operant chamber. The 3-PBC infusions were delivered immediately before the operant session with a Harvard infusion pump in aCSF (composition in mM: NaCl, 120; KCl, 4.8; KH₂PO₄, 1.2; MgSO₄, 1.2; NaHCO₃, 25; CaCl₂, 2.5; d-glucose, 10) as previously described (for details see June et al., 2001).

Histology

After the completion of the behavioral testing, animals were sacrificed by CO₂ inhalation. Cresyl violet acetate (0.20 μl) was injected into the infusion site, and the brains were removed and frozen. The frozen brains were sliced on a microtome at 50 μm sections and the sections were stained with cresyl violet acetate. Infusion sites were examined under a light microscope and indicated on drawings adapted from the rat brain atlas of Paxinos and Watson (1998). Rats with improper placements were excluded from the final data analysis.

Blood EtOH Content (BAC) Determination

To ensure animals were consuming pharmcologically relevant amounts of EtOH during operant sessions, BAC's were collected on all animals on days they did not receive drug treatments using procedures previously described (June et al., 2001).

Statistical Analysis The operant maintained responding data were analyzed by a single factor repeated measures ANOVA with drug treatment (i.e., dose) as the independent factor. The dependent variables were EtOH and saccharin-maintained responding. Each dependent variable was analyzed separately. Post-hoc comparisons between individual drug treatments were made using the Newman Keuls Test in all experiments. In systemic studies, drug treatment comparisons were made against the no injection (e.g., baseline=BL) and Tween-20 vehicle control conditions. In the microinjection studies, drug treatment comparisons were made against the no injection control condition (e.g., baseline=BL) and aCSF control condition. All microinjection data were obtained and analyzed following correct histological verification under a light microscope. To determine the time course of antagonism across the 60 min session, a single factor ANOVA was conducted at each of the six-10 min intervals on the cumulative response data for the respective drug treatment conditions relative to the pooled controlled conditions. EtOH and saccharin maintained responding data were analyzed separately. Post-hoc analyses were performed on the cumulative interval data using the least significant difference (LSD) test. Finally, correlated t-tests were conducted in each experimental group to compare basal response rates between EtOH and saccharin-maintained responding prior to any drug administration.

Results

Chemistry and Molecular Biology Studies

Synthesis of 3-PBC

3-PBC was produced in excellent yield using the more efficient and improved synthesis based on the recently established pharmacophore/receptor model of GABA_{A}-BDZ α1 subtypes (Cox et al., 1998; Huang et al., 2000) (FIG. 1).

Binding Affinity for 3-PBC at Recombinant α1, α2, α3, α5 and α6, Containing GABA_{A} Receptors

Following synthesis, the in vitro binding affinities of 3-PBC, the O-carboline competitive antagonist ZK 93426 (Haefely, 1983; Jensen et al., 1984), the imidazobenzodiazepine competitive antagonist flumazenil [RO15-1788] (Haefely, 1983), and several reference α1 ligands were evaluated at recombinant GABA_{A} receptors as depicted in Table 2. For comparison, data from McKernan et al. (2000) are also shown. The binding affinities were generated using Ltk cells stably transfected with human receptor cDNAs. Portions of these data have recently been reported (Cox et al., 1995). As predicted, the well-known BDZ1 agonists, zolpidem and CL 218, 872 displayed a moderate level of selectivity for the α1 subtype. 3-PBC also displayed a moderate level of selectivity for the α1 subtype, exhibiting a 9.8, 13, and 111 fold selectivity, relative to the α2, α3, and α5 receptors, respectively. 3-EBC, an inverse agonist, (i.e., negative GABA modulator) which was developed in our lab along with 3-PBC displayed a similar, albeit lower selectivity, at the α1 receptor. However, βCCt exhibited the greatest binding selectivity over BZII receptors (α2, α3, and α5) reported to date. βCCt was 3.5 fold more selective than zolpidem and over 20 fold more selective than the antagonist flumazenil at α1 sites. The actions of βCCt on alcohol seeking behavior was recently reported (Carroll et al., 2000; June et al., submitted).

TABLE 2
Displacement of [³H] flunitrazepam in vitro, IC₅₀ nM

| Compound | IC₅₀ nM |
|---|---|
| 3-PBC | 11 |
| 3-EBC | 24 |
| Diazepam | 6 |
| βCCE | 5 |
| Displacement potencies of several 3-substituted β-carbolines and diazepam at wild type BDZ receptors using cerebral cortical membranes. Values represent the mean of | |
| # three or more experiments. S.E.M.s were usually <10%. See Cox et al., (1998) for methodological details. | |

Efficacy of 3-PBC in Modulating GABA at Recombinant α1, α2, α3, and α5 Receptors in the Xenopus Oocytes Assay: Comparison with Other Competitive BDZ Antagonists

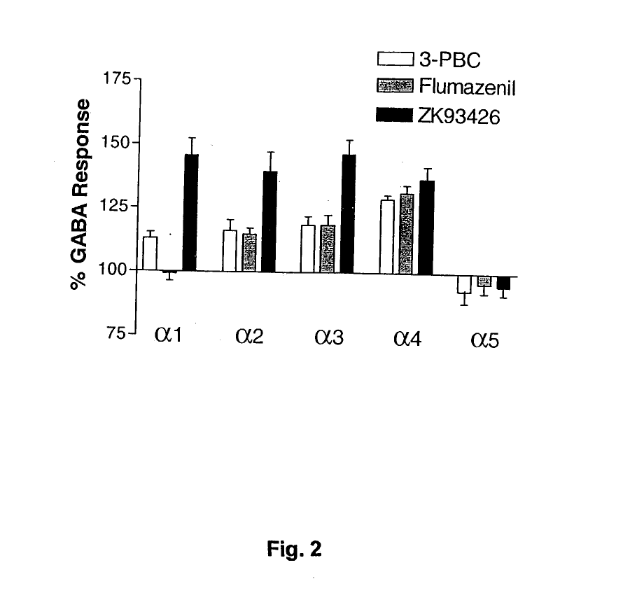

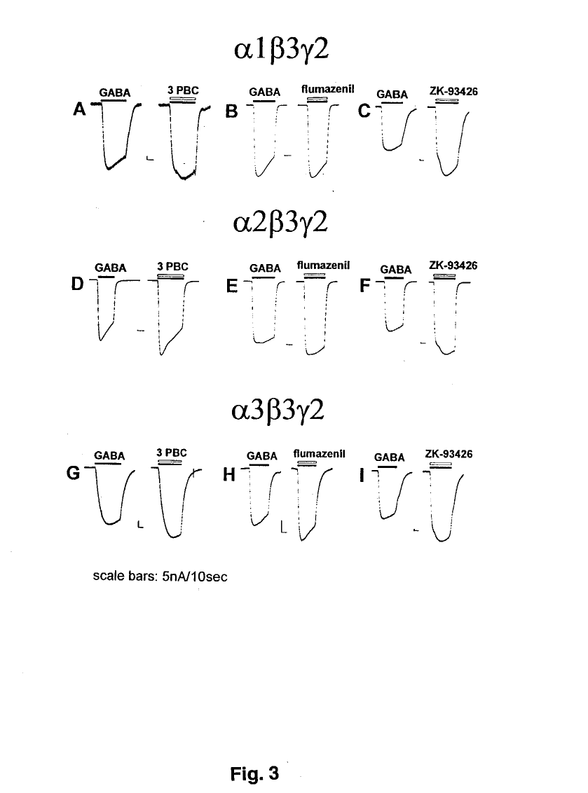

3-PBC's selectivity in relation to physiological efficacy was also determined. For comparison, the activities of the prototypical antagonists ZK 93426 and flumazenil were also evaluated. Receptors comprised of different GABA_{A} α subunits (α1 through α5) were co-expressed with both the β3 and γ2 subunits. To accurately compare modulator activity between receptor subtypes, we utilized an equi-effective (EC₅₀) concentration of GABA for each GABA_{A} receptor subtype: 50 μM for α1β3γ2, 50 μM for α2β3γ2, 30 μM for α3β3γ2, 10 μM for α4β3γ2, and 30 μM for α5β3γ2. All agents were examined at saturating concentrations, either 1 or 10 μM. FIG. 2 shows that 3-PBC acted as a modest positive modulator at α1, α2, α3, and α4 containing receptors (113±4%, 116±7%, 119±6%, 129±3% of GABA response, respectively). At the α1 through α5 receptors, flumazenil exhibited an efficacy profile statistically similar to 3-PBC (P>0.05). At the α1 through α4 receptors, ZK 93426 exhibited a partial to full agonist profile (146+11%, 140±13%, 147±10%, 137±8%, respectively). These effects were statistically greater than 3-PBC and flumazenil at the α1 through α3 receptors (P<0.05). As previously reported (Wafford et al., 1993a,b), flunitrazepam, the full agonist, markedly enhanced GABAergic activity (152±8% to 164±3%) across the α receptors (data not shown). At the α5 receptor, each of the three antagonists exhibited a very weak negative profile which was indistinguishable from each other (P>0.05). The relative magnitude of GABA potentiation for the 3 antagonists across the α1, α2, and α3 receptors is depicted in the traces of FIG. 3. The traces confirm 3-PBC's very weak partial agonist profile, and ZK 93426's moderate level of GABA potentiation across the (α1, α2 and α3 receptor subtypes.

In vitro Binding Affinity of 3-PBC to Rat Synaptosomal Membrane

Since the α1 subtype is most abundant in the rodent cortex (Wisden et al., 1992; Fritschy and Mohler, 1995) the in vitro binding affinities of 3-PBC, 3-EBC, diazepam, and its parent molecule, βCCE were determined in rat cortical membrane (see Table 2). As we have previously demonstrated with the 3-alkoxy series of BDZs (Cox et al., 1998), there was a steady increase in the in vitro potency as chain length was increased from a 3-methoxy moiety (IC₅₀=124 nM) (not shown), to 3-ethoxy (3-EBC) (IC₅₀=24 nM), to a 3-n-propyloxy group (3-PBC) (IC₅₀=11 nM). Thus, the in vitro binding affinity of 3-PBC at synaptosomal cortical membrane was similar to the binding affinity at the recombinant α1 receptor subtype.

Neurobehavioral Studies

Blood EtOH Content (BAC) Determination

Responding for EtOH yielded intakes of 0.67 to 2.85 g/kg of absolute EtOH. EtOH consumption in milliliters was 1.45 to 6.37. BACs ranged from 16-92 mg/DL. BACs correlated significantly with EtOH responding (r=0.77, p<0.01) and intake (r=0.84, p<0.01).

Systemic Injection Studies

Total Session Data

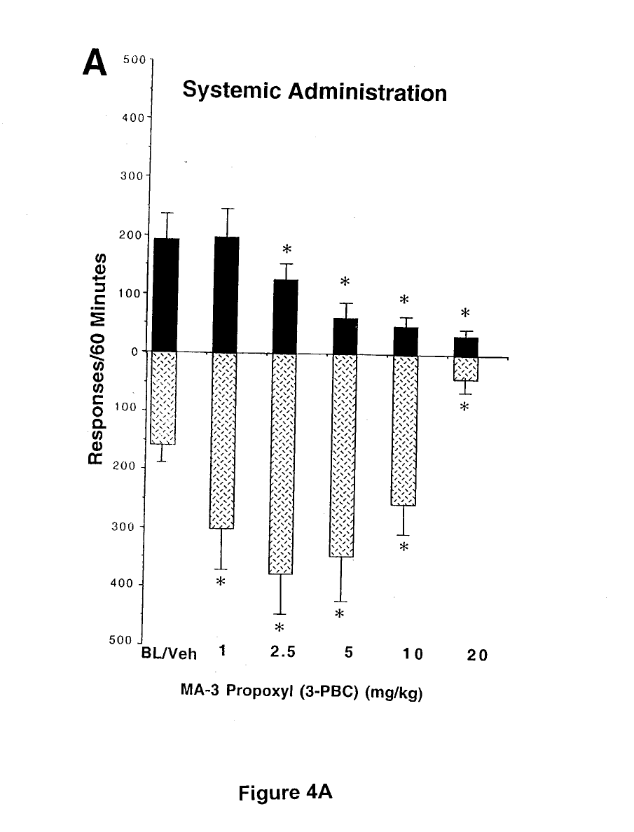

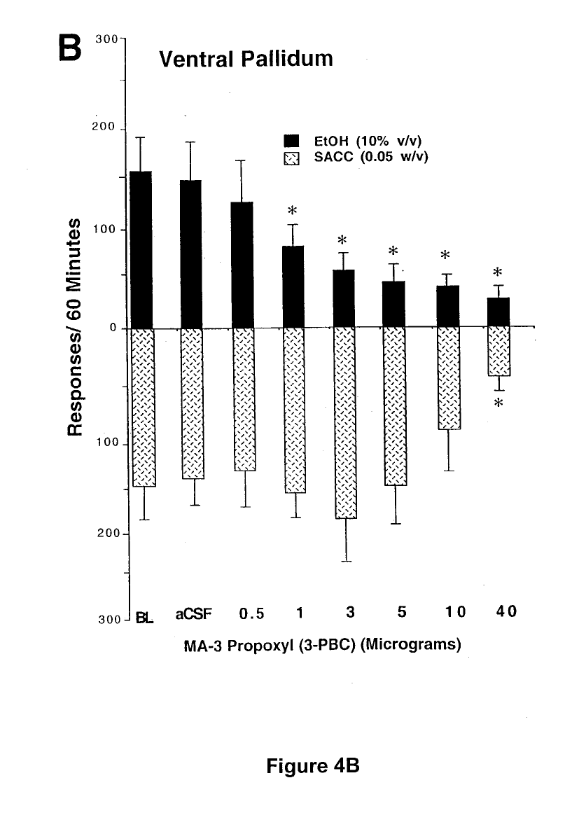

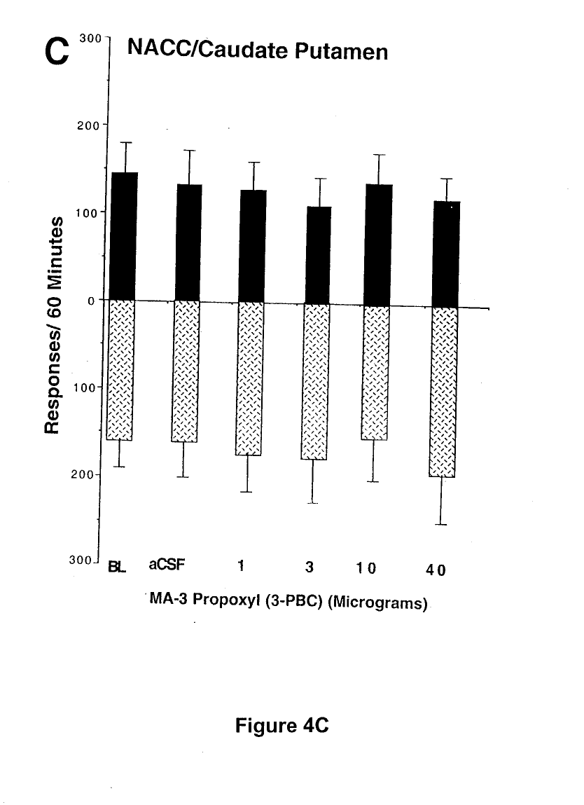

The no injection control and the Tween-20 vehicle conditions were similar (p>0.05), hence; these data were pooled (see FIG. 4A) and used to compare against the drug treatment conditions. FIG. 4A also shows that prior to any drug administration, basal alcohol and saccharin-maintained responding under the control conditions were similar (p>0.05). The top panel of FIG. 4A further shows that 3-PBC produced a significant dose-related reduction on EtOH-maintained responding (p≦0.01). Only the lowest dose failed to significantly reduce responding (p>0.05). These findings yielded a highly significant main effect of drug dose [F_{5.60}=9.827, p<0.01]. The bottom panel of FIG. 4A shows that, in contrast to the effects observed on alcohol responding, 3-PBC produced a significant elevation on responding maintained by saccharin with the 1-10 mg/kg doses (p≦0.05); however, the 20 mg/kg dose significantly suppressed saccharin responding (p<0.01). These data profiles produced a significant main effect of drug dose [F_{5.60}=3.45, p<0.05].

Cumulative within Session Data

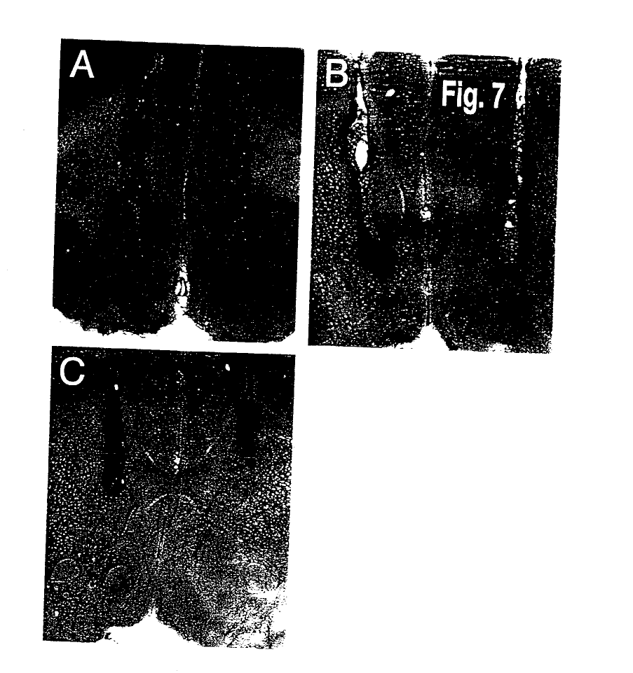

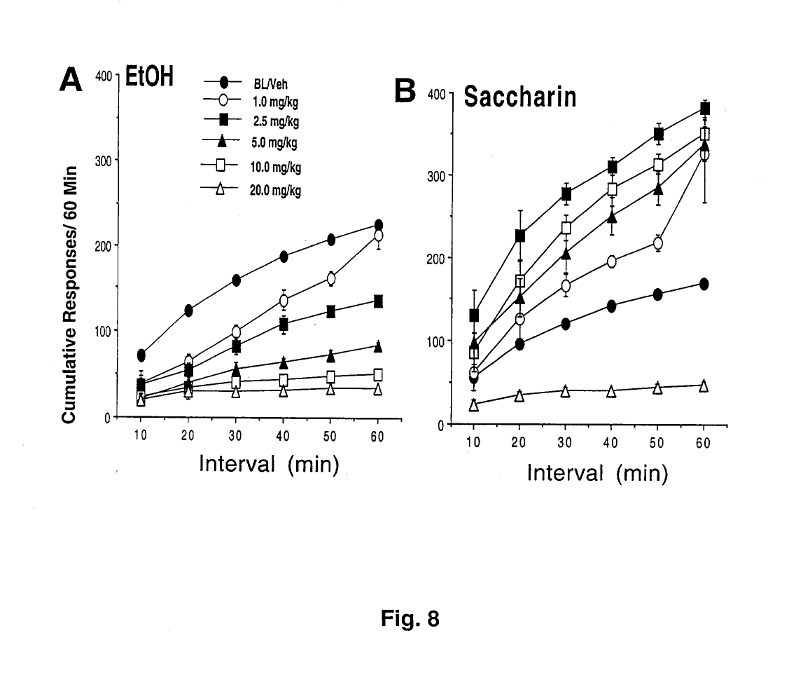

FIG. 8A shows the cumulative within session time course across the 60 min session for alcohol-maintained responding while FIG. 8B shows the cumulative profile for saccharin responding. All 3-PBC doses suppressed the initiation of EtOH responding during the first 10 [F_{5,55}=5.29, p<0.001], 20 [F_{5,55}=4.87, p<0.001] and 30 min [F_{5,55}=3.97, p<0.001] intervals. However, during the latter 40-60 min intervals, only the 2.5-20 mg doses continued to suppressed responding [F_{5,55}=5.72, p<0.001], [F_{5,55}=4.74, p <0.001], [F_{5,55}=5.99, p<0.001], respectively. Post-hoc analyses using the least significant difference (LSD) test confirmed the effects of the individual drug treatment doses at the respective intervals (p<0.05). In contrast to EtOH responding, except for the 20 mg/kg dose, beginning at the 30 min interval, and throughout the remainder of the 60 min session, all 3-PBC doses significantly elevated saccharin-maintained responding [F_{5,55}=4.45, p<0.001], [F_{5,55}=3.84, p<0.005], [F_{5,55}=3.72, p<0.006], [F_{5,55}=4.78, p <0.001], respectively. The 20 mg dose significantly suppressed saccharin-maintained responding beginning at the 20 min interval, and continued throughout the remainder of the 60 min session. These findings were confirmed by post-hoc analyses (p≦0.05).

Microinfusion Studies

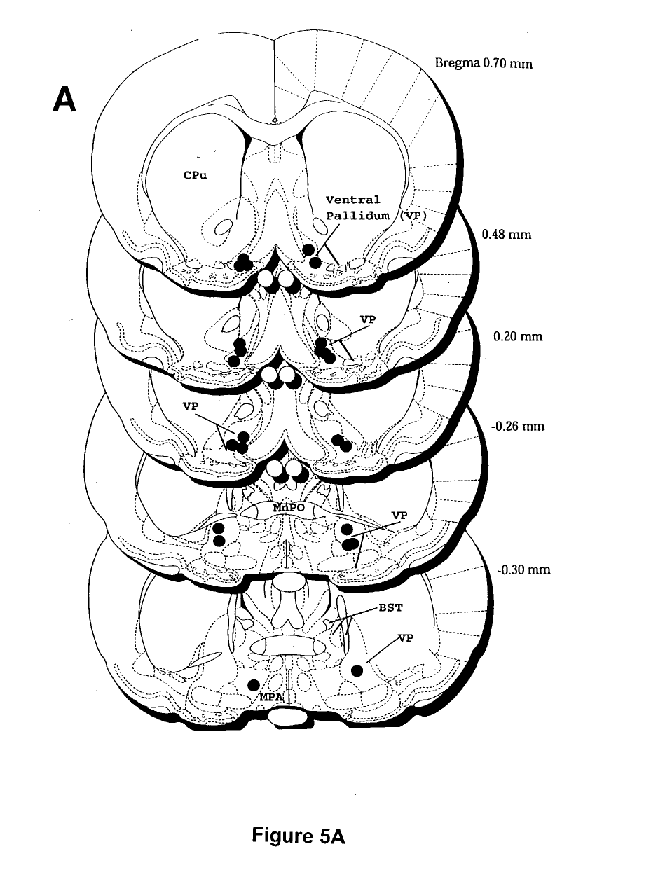

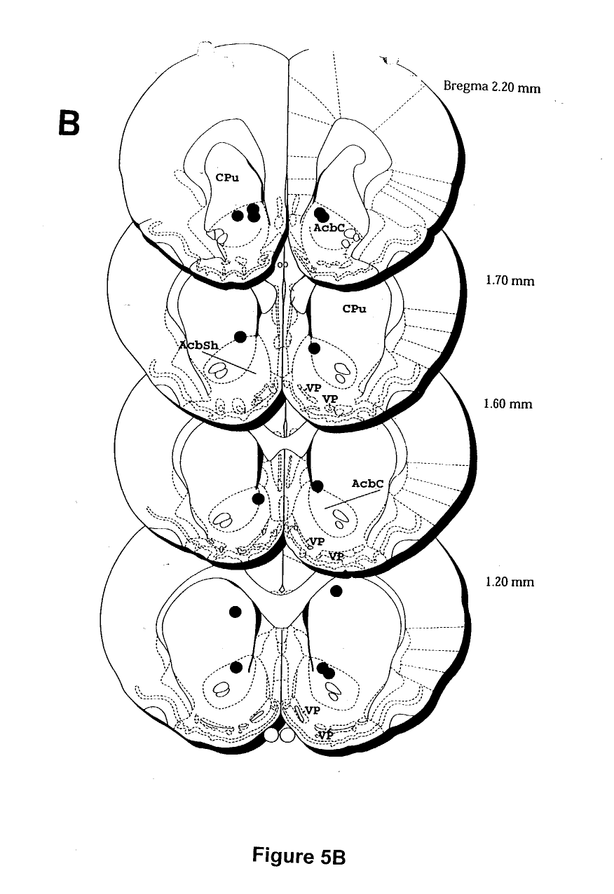

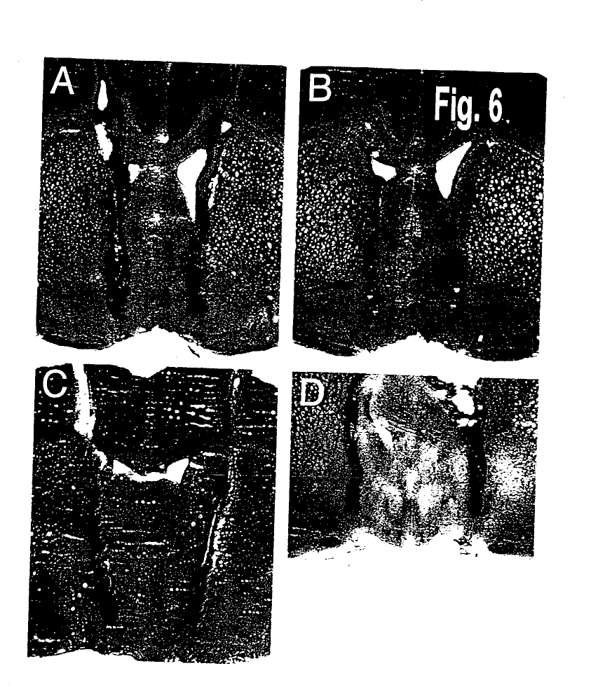

FIG. 5A shows a reconstruction of serial coronal sections of the rat brain illustrating the bilateral guide cannula tips for the correctly implanted subjects (N=12). The histological placements show that the guide cannulas were implanted in the anterior (Bregma 0.70 to 0.20 mm) to medial (Bregma −0.26 to −0.30 mm) VP fields. FIGS. 6A-D depict the actual bilateral placements for “4” of the 12 VP rats in separate photomicrographs illustrating the extent of the lesion sustained as a result of the bilateral guide cannula.

Total Session Data

FIG. 4B shows responding maintained by alcohol and saccharin under the BL and aCSF conditions were similar (p>0.05). Thus, these data were pooled and compared against the 3-PBC dose conditions. 3-PBC dose-dependently reduced alcohol-maintained responding relative to the control condition resulting in a significant effect of drug dose [F_{6,66}=4.43, p<0.02]. Only the 0.5 μg dose failed to significantly reduce alcohol responding (p<0.05). The bottom panel of FIG. 4B reveals that in contrast to the effects observed on alcohol-maintained responding, only the 40 μg dose significantly reduced saccharin responding (p<0.05), however, the overall ANOVA produced a nonsignificant effect of drug dose [F_{6,66}=1.71, P>0.05].

Cumulative Within Session Data

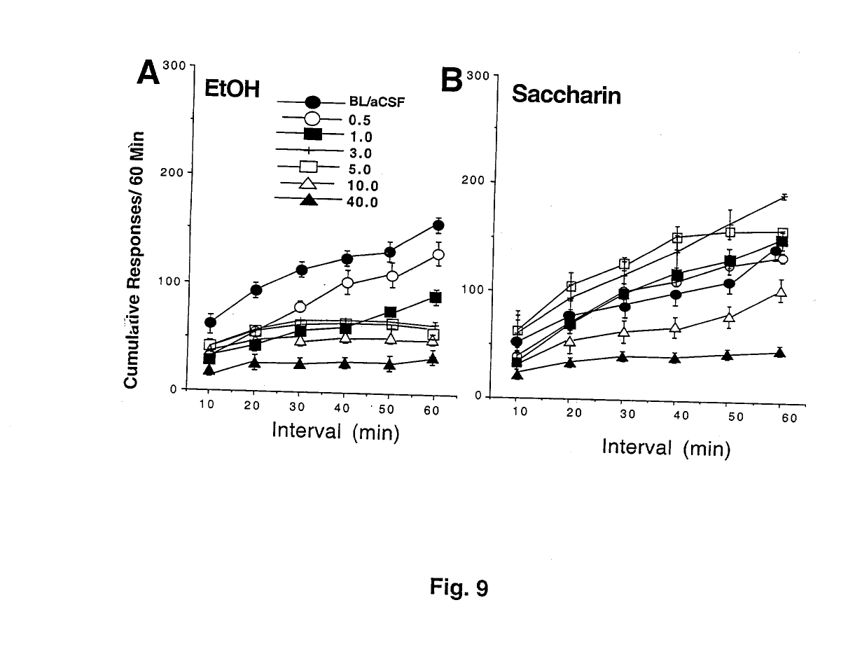

FIG. 9A illustrates the cumulative within session response profile for alcohol under the control and 3-PBC treatments. Similar to the systemic injections, all of the six VP infusions produced a significant reduction on alcohol responding at the initial 10 [F_{6,78}=2.35, p<0.039] and 20 min [F6,78=3.45, p<0.028] intervals. Except for the 0.5 jig dose condition, all 3-PBC infusions continued to suppress EtOH responding at the 30-60 min intervals [F_{6,78}=3.145, p<0.008; F_{6,78}=4.32, p<0.001; F₆,₇₈=4.26, p<0.001; F_{6,78}=4.04, p<0.001; respectively]. In contrast to EtOH maintained responding, with The exception of the 40 μ dose condition, none of the 3-PBC infusions altered responding maintained by saccharin at the 10 min interval (FIG. 9B). The 40 μg dose significantly reduced responding throughout the 10-60 min intervals [F_{6,78}=2.31, p<0.04; F_{6,78}=3.29, p<0.007; F_{6,78}=2.15, p<0.057; F_{6,78}=4.14, p<0.001; F_{6,78}3.36, p<0.006; F_{6,78}=4.36, p<0.001; respectively].

Neuroanatomical Controls

To determine the neuroanatomical specificity of the VP-α1 receptor modulation of alcohol maintained responding, we evaluated 3-PBC's capacity to reduce alcohol-motivated behaviors in the NACC/striatum, a loci reported to be devoid of the (cl receptor subtype (Wisden et al., 1992; Turner et al., 1993; Fritschy and Mohler, 1995; Duncan et al., 1995). FIG. 5B shows a reconstruction of serial coronal sections for the neuroanatomical control rats. The bilateral guide cannula tips for the 7 control subjects were at Bregma 2.20 to Brema 1.20. FIGS. 7A-7C depicts the actual bilateral placements for “3” of the 7 rats in separate photomicrographs illustrating the extent of the lesion sustained as a result of the bilateral guide cannula. FIG. 4C shows rates of responding maintained by EtOH (upper panel) and saccharin (lower panel) following bilateral microinjection of the 5-40 μg doses of 3-PBC. Compared with the pooled aCSF and BL control conditions, the 3-PBC's treatments were without effect on alcohol or saccharin-maintained responding. These findings were supported by a nonsignificant effect of drug treatment for alcohol and saccharin-maintained responding [F_{4,24}=0.365, p>0.05], [F_{4,24}=0.696, p>0.6021], respectively. These data indirectly confirm the topography of the α₁ receptor subtype (Churchill et al., 1991; Duncan et al., 1995) in the striatopallidal area of the P rats.

Discussion

GABA_{A} α₁ Containing Receptors in the VP Exhibit both Reinforcer and Neuroanatomical Specificity in Attenuating Alcohol Motivated Behaviors

To model the human condition of alcohol abuse, we selected as subjects the P rat line. The P rat line has been shown to satisfy all criteria for an animal model of human alcohol abuse (Cicero, 1979; Cloninger, 1987; Lumeng, 1995; McBride and Li, 1998). The overall findings of the present study were that activation of VP-α1 receptors by 3-PBC produced marked reductions on alcohol-maintained responding. These effects were observed in the absence of altering responding for a nondrug reinforcer. 3-PBC's α₁-mediated suppression at the VP level showed a high degree of neuroanatomical specificity. Specifically, the α₁-mediated suppression was not observed with the more dorsal placements in the NACC or caudate putamen. The failure of 3-PBC to alter alcohol self-administration in the NACC/striatum is in agreement with previous research which has consistently reported that the expression of the α1 transcript was not in the NACC and caudate (Churchill et al., 1991; Wisden et al., 1992; Turner et al., 1993; Fritchy and Mohler, 1995; Duncan et al., 1995), as was the magnitude of [³H] zolpidem binding, the α1 selective agonist (Duncan et al., 1995). Criswell and his colleagues (1993; 1995; Duncan et al., 1995) have suggested zolpidem binding sites may be predictive of loci where EtOH activates GABAergic receptors in the CNS.

VP Microinfusion versus Systemic Administration: Comparison of Total Session and Cumulative Time Course Effects

Systemic and VP administration of 3-PBC produced clear dose-dependent reductions on EtOH-motivated responding across a broad range of doses during the 60 min session (FIGS. 8 and 9). In contrast, systemic 3-PBC injections produced marked elevations on responding maintained by saccharin. Such increases are typical of partial and full BDZ agonists (Higgs and Cooper, 1995). However, VP infusions did not alter saccharin responding. It should be recalled that partial agonist effects were observed with 3-PBC at the α₁-α₄ subtypes in the present study (FIG. 2). Hence, it is possible that systemic administration of 3-PBC activates multiple α receptor subtypes, inducing an agonist profile to initiate intake of palatable solutions/general ingesta, while VP infusions results in occupancy of primarily receptors of the α₁ subtype, which is more selective for EtOH. Nonselective GABA agents infused into the VP have been shown to modulate palatable ingesta (Stratford et al., 1999). At the highest tested dose (e.g., 40 μg), VP administration of 3-PBC nonselectively suppressed both EtOH and saccharin responding during the 60 min session. A similar trend was observed with the highest systemic dose (e.g., 20 mg/kg). These nonselective profiles on ingestive responding are likely due to saturation of all α receptor subtypes.

3-PBC disrupted the initiation of alcohol responding during the first 10 and 20 minutes of the operant session and generally led to a gradual attenuation across the 60 min session for some doses, and early termination of responding for others. This pattern was seen for both systemic and VP infusions (FIGS. 8a and 9a). The rapid suppression produced by 3-PBC on EtOH responding is likely due to its high lipophilicity. The 3-propoxy-β-carboline bears one of the most lipophilic substituent to date for BDZs (Huang et al., 2000). In addition, the conversion of the ethyl ester function to the 3-n-propyloxy analog in the synthesis of 3-PBC produced a more water soluble ligand (FIG. 1). This increased water solubility has been shown to be an important factor in increasing the in vivo half-life of BDZs (Cox et al., 1998).

Taken together, the data of the present study provide support for the hypothesis that 3-PBC produces a selective reduction on responding maintained by alcohol, independent of route of administration. Further, the VP α₁ receptor subtype appears to be more salient in regulating alcohol self-administration, while non-al receptors may be more important in the initiation of general ingesta.

GABA VP α₁ Receptors may Interact with Dopamine and Opioid Systems in Regulating Alcohol-Motivated Behaviors

Prior reports have suggested that the VP plays a role in regulating the rewarding properties of psychostimulants and opioids (Austin and Kalivas, 1990; Hubner and Koob, 1990; Hiroi and White, 1993; Gong et al., 1996; Napier and Chrobak, 1992). The VP has also been hypothesized to play some role in regulating alcohol reward because of its location within the mesolimbic circuitry (Samson and Hodge, 1996; Koob, 1999; McBride and Li, 1998). However, this study and the data on βCCt (Example II, infra), another α1 subtype ligand, are the first to directly link this substrate to the rewarding properties of alcohol. It is possible that the VP GABAergic neurons regulate alcohol's euphoric properties via the involvement of GABA within the mesolimbic DA or opioid systems (Austin and Kalivas, 1990; McBride and Li, 1998). The topography of the VP (Phillips and Fibiger, 1991; Kalivas et al., 1993b) places it in a unique position to serve as a pivotal regulator of dopaminergic, opioid and GABAergic inputs that could control EtOH-motivated behaviors.

One hypothesis, albeit somewhat speculative, is that 3-PBC infusions result in activation of GABA α₁ receptors in the VP, and this in turn induces a further enhancement of GABA by activation of α₁ receptors in other subcortical areas (e.g., amygdala, NACC shell, hippocampus, hypothalamus, bed nucleus of the stria terminalis) participating in the rewarding properties of alcohol. The net effect, however, would be an overall increase in GABAergic tone. Kalivas et al., (1993b) contend that increases in GABAergic activity can inhibit inhibitory inputs controlling DA neurons; hence, increases of GABA at the GABA_{A} receptor may disinhibit DA neurons, producing an elevation in DA levels and subsequent reduction in alcohol drinking.

Intrinsic Efricacy: Comparison of 3-PBC with Other Competitive BDZ Antagonists

At saturating concentrations, 3-PBC's efficacy profile was not statistically different from flumazenil across the α subtypes. In contrast, ZK 93426 displayed an efficacy profile similar to that of a full agonist. This finding, that competitive BDZ antagonists are capable of potentiating GABA at some GABA_{A} receptor subtypes in the xenopus oocyte assay, has been observed by previous investigators even at nonsaturating concentrations (Wafford et al., 1993a,b; June et al., 1998c; McKernan et al., 2000). This finding is not surprising as several reports have documented that “some” BDZ antagonists are capable of producing anxiolytic effects in several preclinical models of anxiety (Barret et al., 1985; File and Pellow, 1986; File et al., 1989). Indeed, we have found that both βCCt (Example II) and 3-PBC (unpublished observation) are capable of producing anxiolytic effects in the plus-maze test in selected rodent lines, particularly at moderate to high doses (>7.5 mg/kg).

Similar to 3-PBC, prior reports in our laboratory have suggested that ZK 93426 effectively reduces alcohol-motivated behaviors under a number of experimental conditions (June et al., 1998b). In contrast, flumazenil failed to reliably reduce alcohol-motivated responding (June et al., 1998b). Thus, while flumazenil and 3-PBC's efficacy profile was indistinguishable, their capacity to reduce alcohol motivated responding is substantially different. Moreover, flumazenil was the most α1 selective BDZ antagonist in the Xenopus oocyte assay (FIG. 2). We again concede that binding selectivity, not efficacy, may be the more critical variable in determining whether a BDZ ligand will selectively reduce alcohol motivated behaviors (June et al., 2001).

3-PBC Exhibits Competitive Antagonism in Behavioral Assays

While 3-PBC is capable of producing agonistic effects, it also exhibits a profile consistent with competitive antagonism across several species. In preliminary studies in our laboratory, 3-PBC dose-dependently (7.5 & 15 mg/kg) reversed the suppression produced by chlordiazepoxide (2.5-10 mg/kg) in rats in the absence of intrinsic effects. In the pentylenetetrazole seizure model 3-PBC produced a “neutral” antagonist-like response in mice (Cox et al., 1998). Recently, Rowlett et al., (unpublished observation) using squirrel monkeys demonstrated that 3-PBC was inactive when compared with saline controls in observational studies. In comparison, flumazenil decreased overall activity. In zolpidem discrimination studies Rowlett et al., (submitted) further showed that 3-PBC exhibited properties similar to flumazenil. Specifically, 3-PBC showed surmountable antagonism, characteristic of competitive binding site interaction. Together, these preliminary results demonstrate that 3-PBC is a “neutral” competitive BDZ antagonist with little intrinsic activity in some behavioral paradigms, however, exhibits partial agonist effects in others.

The Utility of BDZ α1 Antagonists in Treating Alcohol Addictive Behaviors

The data of the present study provide support for the hypothesis that GABA_{A}-receptors containing α1 subunits in the VP play an important role in regulating EtOH-seeking behaviors. Under several experimental conditions, the moderately selective (XI subtype antagonist, 3-PBC, reliably and selectively reduced motivated behavior for alcohol. The suppression was observed despite 3-PBC's capacity to display a partial agonistic profile as was seen in vivo and in vitro. We propose that “competitive” BDZ antagonists that exhibit binding selectivity at the α1 subtype, while concurrently displaying a partial agonist efficacy at non-cc subtypes, may have important treatment implications in the design and development of novel pharmacotherapies for alcohol-dependent subjects. Hence, from a clinical perspective, α1 subtype antagonists which are capable of reducing alcohol intake, and concurrently eliminating or attenuating the anxiety associated with abstinence or detoxification, would render them optimal pharmacotherapeutic agents in treating alcohol dependent individuals.

### EXAMPLE II

### Selective Reduction of Alcohol Responding by βCCt

Alcohol addiction and dependence remain a significant public health concern, impacting physical and mental well-being, family structure and occupational stability (Kessler et al., 1997). While advances have been made in the development of novel therapies to treat alcoholism (O'Malley et al., 1992; Volpicelli et al. 1992; Kranzler, 2000; Spanagel and Zieglgansberger, 1997), alcohol-dependent individuals represent a heterogeneous group (Cloninger, 1987; Li et al. 1991; 2000), and it is unlikely that a single pharmacological treatment will be effective for all alcoholics. Hence, a better understanding of the neuromechanisms which regulate alcohol seeking behaviors and the design of clinically safe and effective drugs that reduce alcohol addiction and dependence remain a high priority (Kranzler, 2000; Johnson and Daoud, 2000). While the precise neuromechanisms regulating alcohol-seeking behaviors remain unknown, there is now compelling evidence that the GABA_{A} receptors within the striatopallidal and extended amygdala system are involved in the “acute” reinforcing actions of alcohol (Koob, 1998; Koob et al., 1998; June et al., 1998e; McBride and Li, 1998). The striatopallidal and extended amygdala system include the sublenticular extended amygdala [substantia innominata-ventral pallidum (VP)], shell of the nucleus accumbens, and central nucleus of the amygdala (Heimer et al., 1991; Heimer and Alheid, 1991). Among the potential GABA_{A} receptor isoforms within the VP regulating alcohol-seeking behaviors, GABA_{A} receptors containing the α1 receptor subtype (GABA_{A1}) appear preeminent. Thus, Criswell et al., (1993, 1995) observed that acute alcohol administration selectively enhanced the effects of ionotophoretically applied GABA in the VP. However, no effects were seen in the septum, VTA, and CA1 hippocampus. Further, a positive correlation was observed between alcohol-induced GABA enhancement and [³H] zolpidem binding (an _{Al} subtype selective agonist). Other investigators have identified a dense reciprocal projection from the VP to the NACC (Nauta et al., 1978b; Zahm and Heimer, 1988; Groenewegen et al., 1993), and many of these have been found to be GABAergic neurons (Mogenson and Nielson, 1983; Kuo and Chang, 1992; Churchill and Kalivas, 1994). The NACC is well established as a substrate that regulates the reinforcing properties of abused drugs (Koob, 1998; Koob et al., 1998). Finally, immunohistochemical (Turner et al., 1993; Fritschy and Mohler, 1995) and in situ hybridization studies (Churchill et al., 1991; Wisden et al., 1992; Duncan et al., 1995) have demonstrated that the VP contains one of the highest concentrations of mRNA encoding the _{Al} subunit in the CNS. These findings, together with pharmacological studies suggesting the VP plays a role in reward-mediated behaviors of psychostimulants and opiates (Hubner and Koob, 1990; Napier and Chrobak, 1992; Churchill and Kalivas, 1994; Gong et al., 1996; 1997), led us to hypothesize that the _{A1} containing GABA_{A} receptors regulate alcohol-motivated behaviors.

To test this hypothesis, we developed βCCt, a mixed BDZ agonist-antagonist with binding selectivity at the _{A1} receptor. Behavioral studies in several species (e.g., rats, mice, primates) show that βCCt is a BDZ antagonist, exhibiting competitive binding site interaction with BDZ agonists over a broad range of doses (Shannon et al., 1984; Griebel et al., 1999; Cox et al., 1998; Carroll et al., 2001; Rowlett et al., 2001; Paronis et al., 2001). Other studies show that βCCt produces anxiolytic effects in rodents (Carroll et al., 2001) and potentiates the anti-conflict response induced by Al subtype ligands in primates (Paronis et al., 2001). Thus, βCCt displays an agonist or antagonist profile depending on the behavioral task, species, and dose employed. Studies of recombinant receptors show βCCt exhibits a >10 fold selectivity for the GABA_{A1} over the _{A2} and _{A3} receptors, and a >110 fold selectivity for the _{A1} over the _{A5} subtype (Cox et al., 1995). Hence, βCCt exhibits the greatest binding selectivity of the currently available Al receptor ligands (Sanger et al., 1994; McKernan et al., 2000; Cox et al., 1998).

In the present study, in vitro studies were conducted in recombinant GABA_{A1}-_{A5} receptors in Xenopus oocytes to determine the efficacy of βCCt. Next, a series of in vivo studies were conducted to examine the effects of βCCt, to reduce alcohol responding following parenternal and direct infusions into the VP. The degree of neuroanatomical specificity in modulating alcohol drinking was examined following both bilateral and unilateral control injections of βCCt into the NACC/CPu. The specificity of βCCt on alcohol-induced responding was evaluated by determining its effects in P rats whose response rates for EtOH (10% v/v) and saccharin solutions (0.05% w/v) were similar at basal levels. The effects of βCCt were also examined on a caloric sucrose reward. Finally, since the GABA_{A}_{₁} receptor isoform has recently been implicated in the sedative effects of BDZs (Rudolph et al., 1999; McKernan et al., 2000; Löw et al., 2000), we tested the hypothesis that the GABA_{A}_{₁} receptor played a role in the sedation produced by an intoxicating dose of alcohol (1.25 g/kg). Chlordiazepoxide was used as a reference BDZ agonist. Using the nomenclature recently recommended by the International Union of Pharmacology (IUPHAR), α1-α6 containing GABA_{A} receptors are referred to in the present study as GABA_{A}_{₁}_{-A}_{₆} receptors (Barnard et al., 1998).

Material and Methods

Synthesis of βCCt

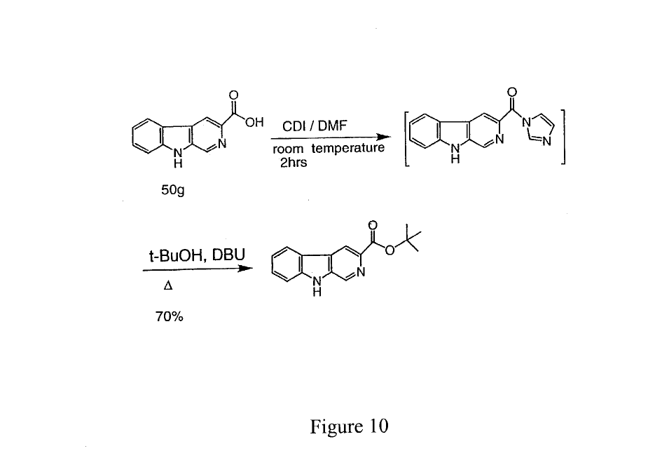

βCCt (β-carboline-3-carboxylate t-butyl ester) [FIG. 10], an _{A1} subtype ligand, was synthesized by modification of the prototypical β-carboline, βCCE, using a previously developed method for t-butyl ester synthesis (see Cox et al., 1995; 1998).

Xenopus Oocyte Expression Studies were performed as previously descrbied for 3-PBC.

Efficacy of βCCt at Recombinant Receptors in the Xenopus Oocyte Assay

The efficacy of βCCt was measured by peak whole cell current responses in the Xenopus oocyte assay to an EC₅₀ concentration of GABA at saturating (1-10 μM) concentrations of the ligand. The efficacies of nonselective, competitive BDZ antagonists [e.g., RO15-1788, ZK 93426) were also evaluated.

Subjects

General

The alcohol-preferring (P) and high-alcohol drinking (replicate line #1) (HAD-1) rats were used to model the human condition of alcohol abuse; both rat lines are accepted as animal models of chronic alcohol seeking behavior in humans to the satisfaction of the alcohol research community (Cloninger, 1987; Lumeng, 1995; McBride and Li, 1998). Rats were obtained from the Alcohol Research Center at Indiana University School of Medicine. Female P and HAD-1 rats were used in all experiments; however, due to the large number of P (n=128 rats) and HAD-1 (n=98) rats within the experimental design (Total N=226), it was not possible to obtain all P or HAD-1 rats of a single generation. Animals were individually housed in wire-mesh stainless steel cages or plastic tubs. The vivarium was maintained at an ambient temperature of 21° C. and was on a normal 12 hr light/dark cycle. All rats were provided ad libitum access to food and water. The sole exception was the rats of the operant self-administration studies wherein rats were fluid deprived 23 hr daily (see below) during the first 2 days of the training phase. Thereafter, these animals were maintained on ad libitum food and water. All training and experimental sessions for all subjects took place between 9 a.m. and 3 p.m. The treatment of rats for all studies was approved by the institutional review board within the School of Science at IUPUI. In addition, all procedures were conducted in strict adherence with the NIH Guide for the Care and Use of Laboratory Animals.

Operant Self-administration Studies

Systemic and Ventral Pallidum (VP)

Female P rats (N=35) from the S48 and S49 generations and female HAD-1 rats (N=24) from the S34 generation were used in the present study. Animals were approximately 3-4 months of age and weighed between 209 and 384 g at the beginning of the experiment. No effects of estrous cycle have been observed on drinking patterns in genetically selected rats (McKinzie et al., 1996), and female P rats maintain their body weights within a range that allows for more accurate stereotaxic placement than male P rats (Nowak et al., 1998; June et al., 2001).

Locomotor Sedation Studies

Female P rats (N=42) from the S50 and S51 generations and female HAD-1 rats (N=42) from the S35 generation were used. Rats were approximately 3 months of age and weighed between 225 and 310 g at the beginning of the experiment.

Drug and Solutions

βCCt

For systemic drug administrations, βCCt was prepared as an emulsion in a Tween-20 solution (Sigma Chemical Co., St. Louis, Mo.) that was comprised of 100 mls of a 0.90% sodium chloride solution and two drops of Tween-20. βCCt was sonicated (Fisher Scientific, Springfield, N.J.) to aid in dissolving the compound. Tween-20 vehicle solution was administered as the control injection for all systemic experiments. Systemic drug injections were given intraperitoneally (i.p.) in an injection volume of 1 ml/kg. For the microinjection studies, βCCt was dissolved in artificial cerebrospinal fluid (aCSF) (see below).

Other Drugs and Solutions

EtOH (10% v/v) [USP], saccharin (0.05% w/v), and sucrose (1% w/v) [Fisher Scientific] solutions were prepared for the operant chamber as previously described for oral self-administration (June et al., 1998b; 1998d; 1998e). Chlordiazepoxide was obtained from RBI, (Natick, Mass.) and mixed in sterile saline (0.9%) for i.p. injections in the locomotor sedation studies. EtOH (10% v/v) was also mixed in sterile saline for i.p. injections in the locomotor sedation studies. A volume sufficient to produce a 1.25 g/kg EtOH dose was employed. The competitive BDZ antagonists ZK 93426 (Schering, Berlin, FRG), flumazenil (Ro15-1788) and the inverse agonist Ro15-4513 (Hoffman La Roche, Nutley, N.J.) were donated as gifts for use in the Xenopus oocyte studies.

Neurobehavioral Studies

Operant, Self-administration Apparatus, Training, and Drug Treatment Procedures Behavioral Testing Apparatus. Behavioral testing was conducted in 15 standard operant chambers (Coulbourn Instruments, Allentown, Pa.) equipped with two removable levers and two dipper fluid delivery systems enclosed in sound-attenuated cubicles as previously described (June et al., 1998b; 1998e). All dipper presentations provided a 1.5-sec access to a 0.1-ml dipper, followed by a 3-sec time out period. Above each lever, three stimulus lights (red, green and yellow) were present, and a stimulus delivery/reinforcer was indicated by illumination of the middle (green) stimulus light. Responses and reinforcements were recorded and controlled by a Dell computer using the 4.0 Coulbourn L2T2 operant software package.

Training Phase

P Rats. A concurrent fixed-ratio schedule was employed to investigate the capacity of systemic and direct microinjections of βCCt in the VP to modify EtOH and saccharin-maintained responding in P rats. The specific details of these procedures have recently been described (June et al., 1998e; 2001). In brief, rats were initially trained to orally self-administer EtOH and water in daily 60 min sessions on a concurrent FR1 schedule in a two-lever choice situation. After a period of stabilization on the FR1 schedule, the response requirement was increased to a concurrent FR4 schedule and the water reinforcer was replaced with saccharin (0.025-0.05% w/v). The importance of alternative and concurrently presented reinforcers in examining the positive reinforcing properties of drugs of abuse has previously been discussed (Meisch and Lemaire, 1993; June in press).

HAD-1 Rats. To assess the capacity of βCCt to modulate EtOH responding in the VP, rats were trained to lever press for alcohol only (see below). However, an alternate-day access paradigm was employed to investigate the capacity of systemic injections of βCCt to modify EtOH and sucrose-maintained responding. These schedules were employed as previous research has demonstrated that unlike P rats, HAD-1 rats show a profound reduction in EtOH intake (Lankford et al., 1991; Lankford and Myers, 1994) and responding (June in press) when presented concurrently with a palatable nondrug reinforcer. In the VP study rats were initially trained to orally self-administer EtOH and water in daily 60 min sessions on a concurrent FRI schedule in a two-lever choice situation. After a period of stabilization on the FRI schedule, the response requirement was increased to a concurrent FR4 schedule and the water reinforcer was gradually eliminated from the protocol and replaced with EtOH. Thus, the VP HAD-1 rats responded concurrently for EtOH solutions on both levers. The VP HAD rats were then stabilized on this regimen for 3 weeks prior to any microinfusions. Responding was considered stable when responses were within ±20% of the average responses for five consecutive days.

Rats in, the systemic study were trained in a similar manner as the VP animals, except that after a 2 week stabilization period on the concurrently presented EtOH solutions, a series of preliminary studies were conducted to determine the sucrose concentration that produced response rates and profiles similar to that of EtOH during an alternate-day presentation schedule. Following this determination, rats were then stabilized on a regimen of 10% (v/v) EtOH on day 1, and 1% (w/v) sucrose on day 2. This alternate day paradigm continued until a two weeks. After this final stabilization period, the drug treatment phase began. Again, responding was considered stable when responses were within ±20% of the average responses for five consecutive days. The position of the levers and associated dippers for each reinforcer was alternated daily to control for the establishment of lever preference under all concurrent schedules when two different reinforcers were present.

Systemic Drug Treatment Procedures

βCCt was administered 15 min before the operant session to allow for optimal absorption and CNS distribution. βCCt was tested at doses of 1-40 mg/kg. The duration of the operant sessions was 60 min, however, subjects were tested at 24 and 48 hours post-drug administration to determine if any residual drug effects remained. A minimum of 72 and maximum of 96 hours was allocated between drug treatments to permit animals to return to baseline levels. This period prevented confounding of drug treatments due to residual effects. The HAD-1 rats were tested at lower doses since our preliminary studies indicated that the dose-response curve was much lower in the HAD line compared with the P rats. All systemic drug treatments were given in a randomized design.

Surgery

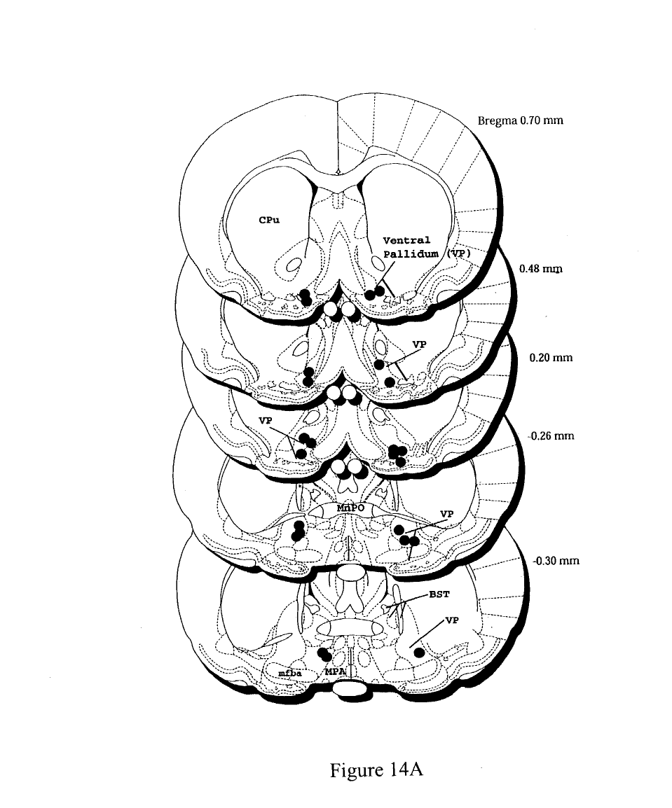

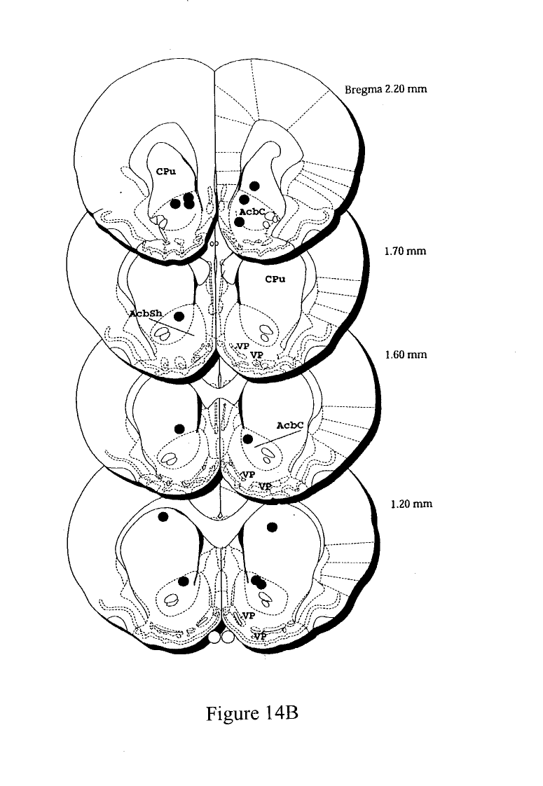

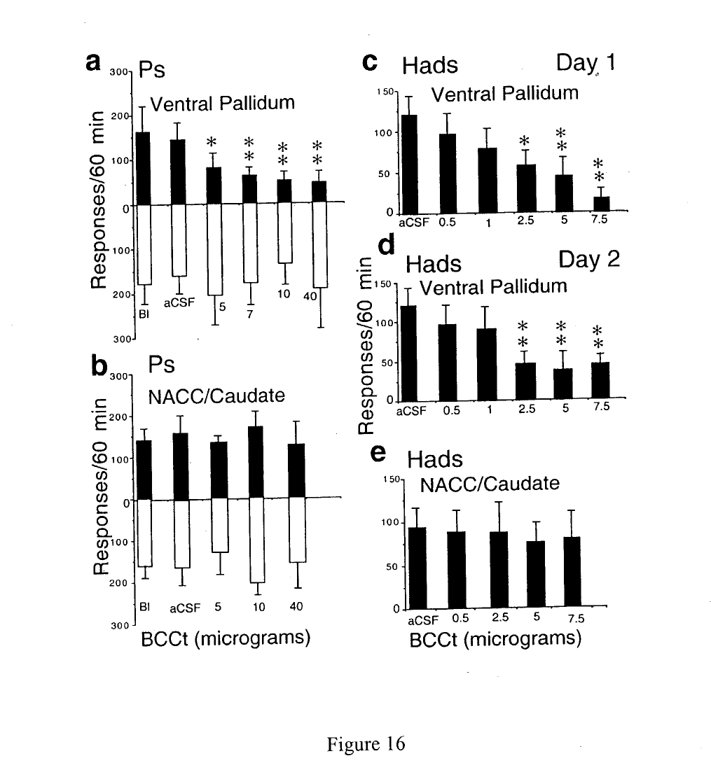

Guide cannulae were stereotaxically implanted bilaterally in the anterior (AP +0.48 mm; ML ±1.6 mm; DV −8.2 mm, with 6° lateral angle) [P rat n=7] and medial (AP −0.26 mm; ML ±2.5 mm; DV −8.0 mm) [P rat n=7; HAD-1 rat n=]J] VP according to the Paxinos and Watson (1998) atlas. The neuroanatomical control rats were implanted in either the CPu or NACC. The coordinates for the CPu rats [P rat n=3; HAD-1 rat n=4] were AP +1.5; ML ±2.5; DV −4.2. In the NACC group, rats were implanted in the shell (AP +1.4; ML ±0.8; DV −6.0) [P rat n=3; HAD-1 rat n=4] or core (AP +1.4; ML ±1.7; DV −5.7) [P rat n=3; HAD-1 rat n=3]. Thus, a total of 14 P rats were bilaterally implanted with a cannula in the left and right VP. While a total of 9 control P rats were bilaterally implanted with cannulas in the left and right NACC or CPu. A total of 11 HAD-1 rats were unilaterally implanted in the left hemisphere with the guide cannula aimed at the VP, and unilaterally implanted in the right hemisphere with a the guide cannula aimed at the CPu/NACC. This strategy was employed to further substantiate the neuroanatomical specificity of the _{A1} receptor subtype in the ventral striatopallidal area in regulating alcohol-motivated behaviors. In experimental and control animals, the cannulas were aimed 1 mm above the intended brain loci. A stylet which protruded 1 mm beyond the tip of the guide cannulae was inserted when the injector was not in place. The sample sizes obtained after the brains were evaluated under the light microscope for correct cannula placements reflect the data shown in FIGS. 14 and 16.

Microinfusion Procedures

The microinfusions were delivered immediately before the operant session with a Harvard infusion pump, during which time animals were able to move about freely in their home cages (for details see June et al., 2001). The injection cannula extended 1 mm beyond the tip of the guide cannulae. βCCt was dissolved in artificial cerebrospinal fluid (aCSF) (composition in mM: NaCl, 120; KCl, 4.8; KH₂PO₄, 1.2; MgSO₄, 1.2; NaHCO₃, 25; CaCl₂, 2.5; d-glucose, 10). When necessary, HCl acid or NaOH was added to the solutions to adjust pH levels to ˜7.4±0.1. βCCt was infused bilaterally in P rats for 5 min at a rate of 0.1 μl/1 min using a 28 gauge injector cannulae. HAD-1 rats were infused unilaterally at a similar rate. Each injector cannula was connected by polyethylene tubing to a 10 μl Hamilton microsyringe. The injection volume delivered to each hemisphere was 0.5 μl, with a total injection volume for both the left and right hemispheres for both P and HAD-1 rats of 1.0 μl. All aCSF and drug treatments were administered in a randomized design in all experiments. P rats received a maximum of 7 bilateral infusions, while HAD-1 rats received a maximum of 7 unilateral infusions in one hemisphere and 7 in the other.

Histology

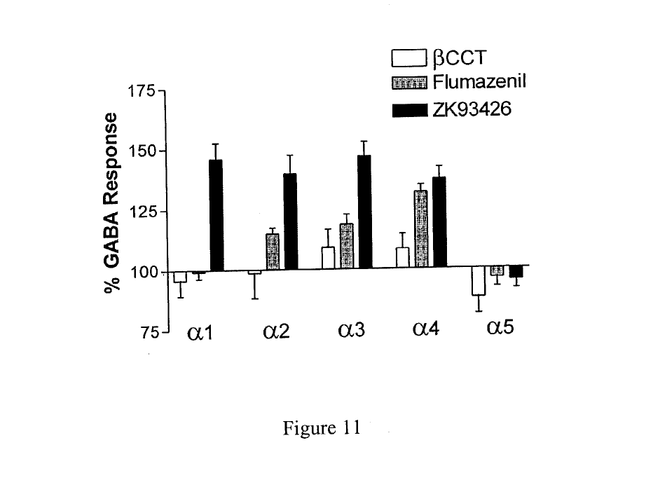

After the completion of the behavioral testing, animals were sacrificed by CO₂ inhalation. Cresyl violet acetate (0.50 μl) was injected into the infusion site, and the brains were removed and frozen. The frozen brains were sliced on a microtome at 50 μm sections and the sections were stained with cresyl violet. Infusion sites were examined under a light microscope and indicated on drawings adapted from the rat brain atlas of Paxinos and Watson (1998). Only rats with correct cannula placements were used in the final data analysis. A reconstruction of serial coronal sections of the rat brains for P and HAD-1 rats used in the data analysis is depicted in FIGS. 10-11. The coronal sections show the guide cannulas were implanted in the anterior (Bregma 0.70 to 0.20 mm) to medial (Bregma −0.26 to −0.30 mm) VP fields, while the control placements were located more dorsally in the NACC and CPu areas.

Statistical Analysis

The operant maintained responding data were analyzed by a single factor repeated measures ANOVA with drug treatment (i.e., dose) as the independent factor. In the systemic studies, the dependent variables were EtOH and saccharin-maintained responding in the P rats (N=11) and EtOH and sucrose-maintained responding for the HAD-1 rats (N=11). In the microinfusion studies, the dependent variables were EtOH and saccharin-maintained responding for the P rats (VP: N=11; NACC/CPu: N=7) and only EtOH in the HAD-1 rats (VP: N=9; NACC/CPu: N=9). Each dependent variable was analyzed separately. Post-hoc comparisons between individual drug treatments were made using the Newman Keuls Test in all experiments. In the concurrent and alternate day schedules, correlated t-tests were used to confirm that response rates for EtOH and saccharin/sucrose responding under baseline and aCSF conditions were similar.

Locomotor Sedation Study

Apparatus

Ambulatory count in the open field was recorded individually for 10 min in a plexiglas chamber (42 cm×42 cm 30 cm) using a Digiscan activity monitoring system (Acuscan Electronics, Columbus, Ohio, USA) (for details of the monitoring system see June et al., 1998d).

Systemic Injection Procedures

βCCt (15 mg/kg i.p.) and chlordiazepoxide (10 mg/kg i.p.) were administered 15 and 30 min, respectively, prior to the rats being placed in the open field. EtOH (1.25 g/kg) was given 5 min prior to placing the rats in the open field. When βCCt was given in combination with either chlordiazepoxide or EtOH, it was given 10 min prior to chlordiazepoxide and EtOH. As noted above, βCCt was administered in Tween-20 solution, while all other drugs were mixed in sterile saline. Animals were tested between 9 am-3 pm.

Statistical Analysis for Interactional Studies

HAD-1 and P rats were randomly assigned to each drug treatment group. A between group ANOVA with drug treatment (i.e., dose) as the independent factor was conducted for HAD-1 (n=6-9 per treatment group) [total N=42] and P (n=6-8 per treatment group) [total N=42] rats on the locomotor activity parameter (i.e., ambulatory count). Post-hoc comparisons between individual drug treatment groups were made using the Newman Keuls Test in all experiments.

Results

Efficacy of βCCt in Modulating GABA at Recombinant GABA_{A}_{₁}_{-A}_{₅} Receptors in Xenopus Oocytes: Comparison with other BDZ Antagonists

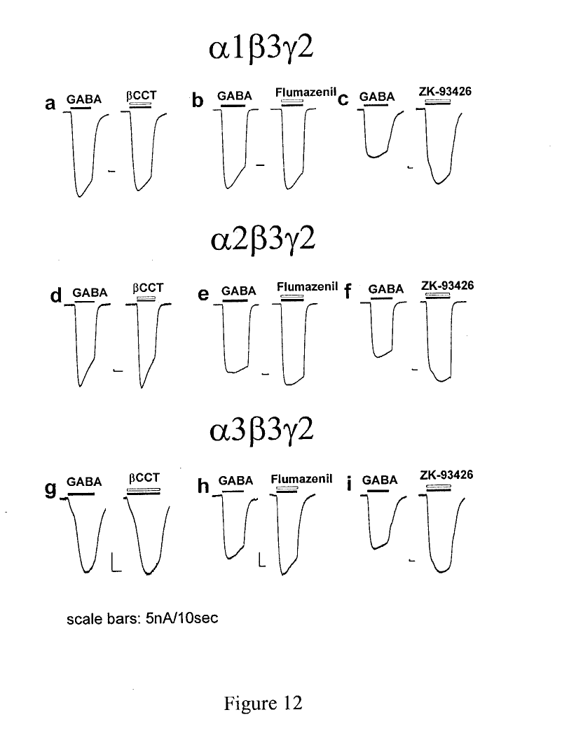

βCCt exhibited either a neutral or low efficacy agonist response at GABA_{A}_{₁} (96±7%) _{A}_{₂} (99±10%), _{A}_{₃} (108±6%), and _{A}_{₄} (107±5%) receptors (FIG. 11). However, a low efficacy partial inverse agonist response was observed at the _{A}_{₅} receptor (88±7% of the GABA response). Flumazenil exhibited an efficacy profile that was qualitatively similar to βCCt at the _{A}_{₁} (99±5%), _{A}_{₃} (118±7%), and _{A}_{₅} (96±6%) subtypes. At the _{A}_{₂} receptor, flumazenil produced a low efficacy agonist response (115±4%) while βCCt was GABA neutral (98±10%). Flumazenil also produced a qualitatively similar response to βCCt at the _{A}_{₄} receptor albeit, the magnitude of GABA potentiation by flumazenil far exceeded that of βCCt (132±6% vs 108±6%, respectively). In contrast, ZK 93426 produced a clear agonist profile, potentiating GABAergic activity by 137±8% to 148±11% across the _{A}_{₁} to _{A}_{₄} subtypes, but was GABA neutral at the _{A}_{₅} receptor (˜96±6%). FIG. 12a-i depicts the current traces illustrating the relative magnitude of GABA potentiation by βCCt, flumazenil and ZK 93426. Despite the qualitatively similar response profile of βCCt and flumazenil, the traces clearly reveal that βCCt did not remarkably effect the GABA currents at the _{A}_{₁}, _{A}_{₂}, _{A}_{₃} or _{A}_{₄} (data not shown) subtypes relative to the control condition. In contrast, flumazenil significantly increased the GABA currents at the _{A}_{₂} (P<0.09), _{A}_{₃} (P<0.05), and _{A}_{₄} (P<0.01) subtypes relative to the control condition. The traces also confirmed the marked potentiation of the GABA current by ZK 93426 at the _{A}_{₁} to _{A}_{₃} and _{A}_{₄} (data not shown) subtypes compared with the control condition (p<0.01).

Neurobehavioral Studies

Blood EtOH Content (BAC) Determination

Body weights of the P (N=10) and HAD-1 (N=9) rats used for BAC determination ranged from 290 to 407 grams. BACs were collected on days that no drug treatments were administered in both rat lines. EtOH responding for P rats yielded intakes of 0.67 to 2.78 g/kg of absolute EtOH. Consumption in milliliters was 1.45 to 6.37. BACs ranged from 16-92 mg/dl. BACs correlated significantly with EtOH responding (r=0.78, p<0.01) and intake (r=0.82, p<0.01). For the HAD-1 rats, alcohol responding yielded intakes of 0.43 to 1.94 g/kg of absolute EtOH. EtOH consumption in milliliters was 0.84 to 5.34. BACs ranged from 12-86 mg/dl. BACs correlated significantly with EtOH responding (r=0.68, p<0.05) and intake (r=0.66, p<0.05).

Parenternal Injection Studies

P Rats

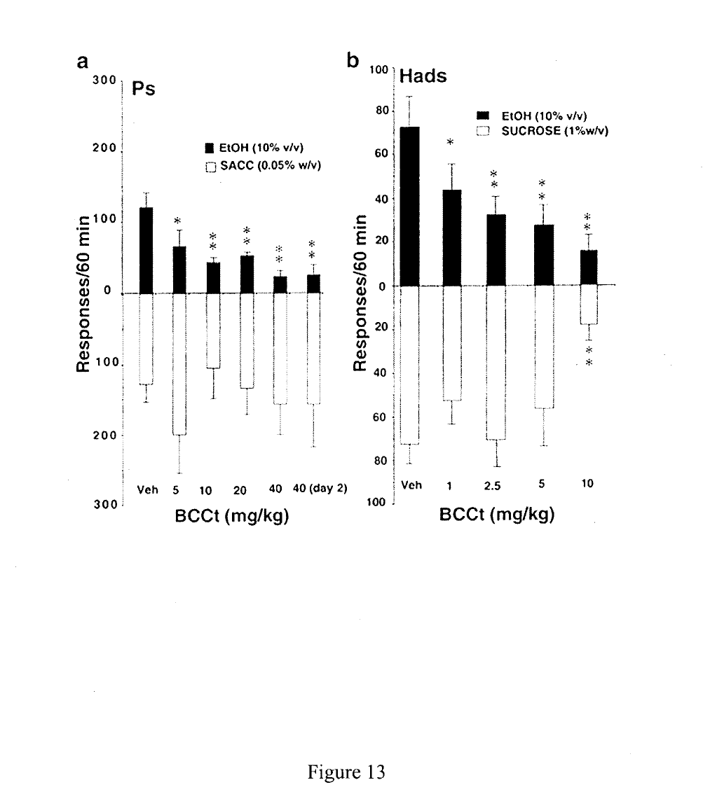

FIG. 13a shows basal operant response rates for EtOH were within 92% of response rates for saccharin (P>0.05). The 5-40 mg/kg βCCt treatments suppressed responding maintained by alcohol yielded a significant main effect of dose [F(5, 45)=4.64, p<0.01]. The Newman Keuls post-hoc tests revealed that all doses significantly suppressed alcohol-maintained responding compared with the control condition (p<0.05). Twenty-four hr post-drug administration, the 40 mg/kg dose continued to suppress responding by 76% of control levels (p<0.05). In contrast to the effects on alcohol-maintained responding, βCCt was without effect on responding maintained by saccharin [F(5, 45)=1.64, p>0.05].

HAD-1 Rats

FIG. 13b shows basal operant response rates for EtOH and sucrose were very similar (P>0.05). The 1-10 mg/kg βCCt injections dose-dependently suppressed responding maintained by alcohol yielding a significant main effect of dose [F(4, 40)=7.84, p<0.001]. The Newman Keuls post-hoc tests confirmed that all doses significantly suppressed alcohol-maintained responding compared with the control condition (p<0.05). The bottom panel of FIG. 13b shows that βCCt suppressed responding maintained by sucrose only with the 10 mg/kg dose, however, the overall ANOVA failed to reached statistical significance [F(4, 40)=1.64, p>0.05]. Subsequent post-hoc test confirmed that the 10 mg/kg dose produced a marked suppression on responding maintained by sucrose (p<0.01).

Microinfusion Studies

P Rats

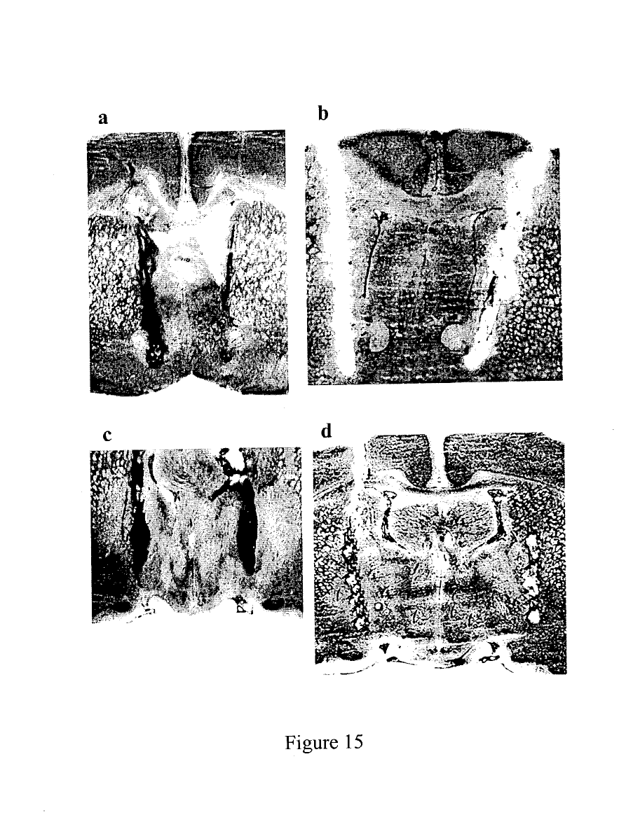

FIG. 14a shows a reconstruction of serial coronal sections of the rat brain illustrating the bilateral guide cannula tips for the 12 VP rats with correct cannula placements. The histological placements show that the guide cannulas were implanted in the dorsal (Bregma 0.70 to 0.20 mm) to medial (Bregma −0.26 to −0.30 mm) VP field, with one rat implanted in the posterior VP (Bregma −0.80). FIGS. 15a-d depict the actual bilateral placements for 4 of the 12 VP rats in separate photomicrographs illustrating the extent of the lesion sustained as a result of the bilateral guide cannula. The top panel of FIG. 16a shows behavioral data for the P rats bilaterally infused with βCCt (5-40 μg) compared with the no injection baseline (i.e., BL) and the artificial cerebral spinal fluid (aCSF) control conditions. Responding maintained by EtOH and saccharin under the BL and aCSF conditions were similar (p>0.05). Thus, these data were pooled and compared against the βCCt conditions. βCCt dose-dependently reduced EtOH-maintained responding relative to the control conditions resulting in a significant effect of drug dose [F(4, 44)=4.36, p<0.05]. Post-hoc analyses confirmed that all βCCt doses significantly reduced EtOH responding (p<0.05). The bottom panel of FIG. 16a shows data for saccharin-maintained responding after the βCCt and control infusions. In contrast to the effects observed on EtOH-maintained responding, βCCt did not alter responding maintained by saccharin with any of the tested doses and produced a nonsignificant effect of drug dose [F(4, 44)=1.58, p>0.05].

A reconstruction of serial coronal sections for the neuroanatomical control rats is depicted in FIG. 14b. The bilateral guide cannula tips for the 7 control subjects were at Bregma 2.20 to Bregma 1.20. FIG. 16b shows rates of responding maintained by EtOH (upper panel) and saccharin (lower panel) following bilateral microinjection of the 5-40 μg doses of βCCt. Compared with the aCSF and BL control conditions, none of the βCCt treatments altered EtOH or saccharin-maintained responding. These findings were supported by a nonsignificant effect of drug treatment for EtOH and saccharin-maintained responding [F(4, 24) 0.299, p>0.05], [F(4, 24)=1.84, p>0.05], respectively.

HAD-1 Rats

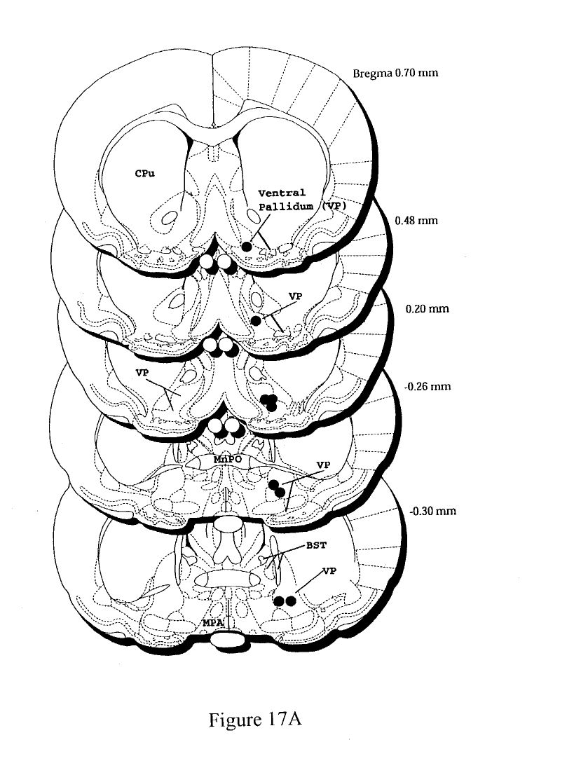

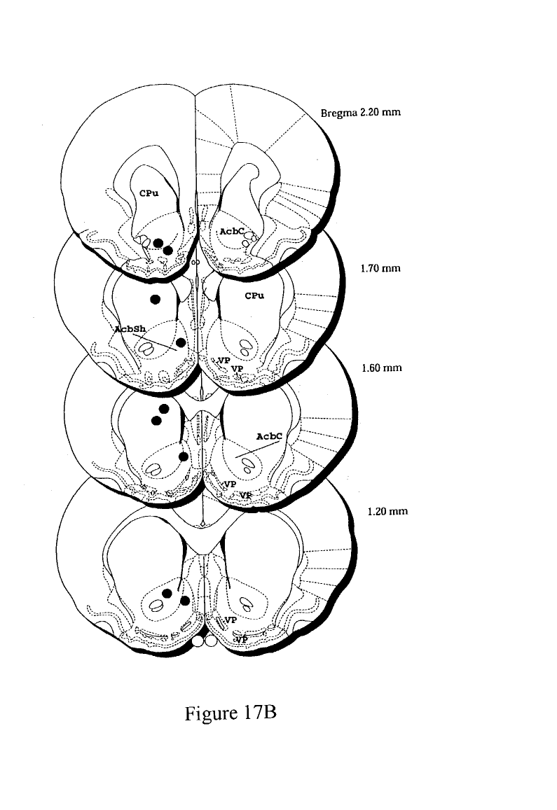

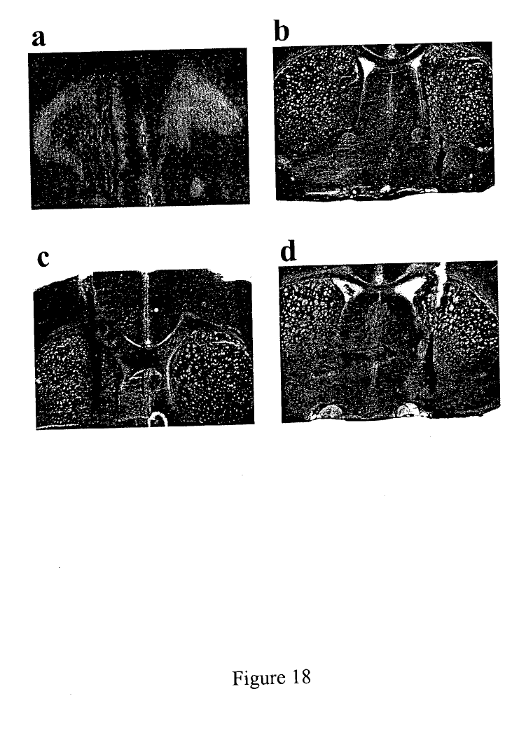

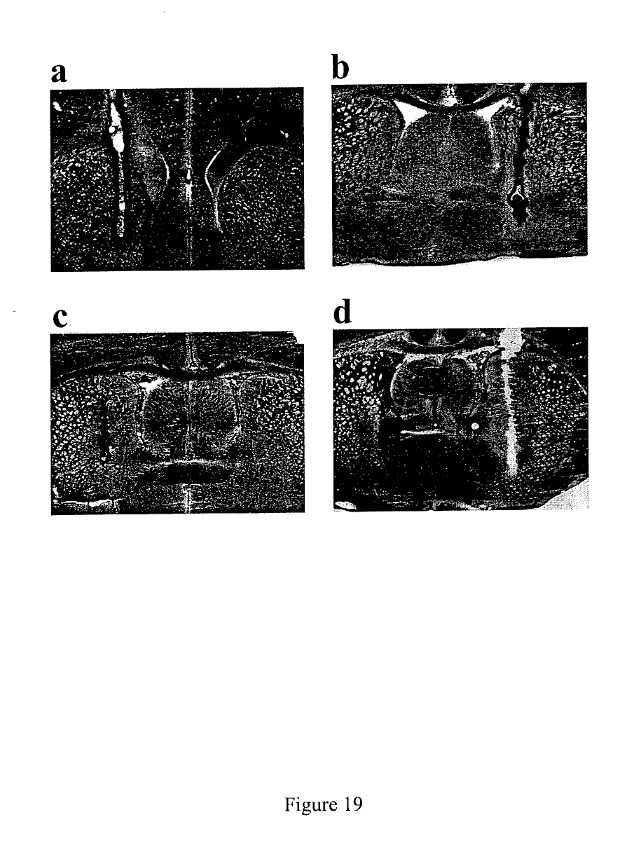

As noted above, HAD-1 rats were trained to lever-press for alcohol only. In addition, to further substantiate the neuroanatomical specificity of the _{A1} receptor, HAD-1 rats received a unilateral implant in the VP and a second implant in either the CPu or NACC. Of the 11 rats implanted, 9 were implanted in both the VP and CPu/NACC areas. FIG. 17a-b shows a reconstruction of serial coronal sections for the rats with the correct placements in both loci. The VP cannulae were at Bregma 0.48 to −0.80 (FIG. 17b) while the CPu/NACC implants were more dorsal at Bregma 2.20 to −0.26 (FIG. 17a). FIGS. 18a-b, 18c-d, 19a-b, 19c-d, depict representative photomicrographs for four rats, with each having a single cannula track in the CPu/NACC and the other in the anterior to posterior VP. FIG. 16c shows rates of responding maintained by EtOH following unilateral microinjection of the 0.5-7.5 μg doses of βCCt into the VP of HAD-1 rats. Compared with the aCSF control condition, βCCt dose-dependently reduced EtOH responding and yielded a significant effect of drug dose [F(5, 40)=4.315, p<0.003]. However, post-hoc analyses showed that only the 2.5-7.5 μg doses significantly reduced responding (p≦0.05). FIG. 16d shows that twenty-four hr post-drug administration the 2.5-7.5 μg doses continued to reduce responding by as much as 54-63% of control levels [F(5, 40)=4.91, p<0.001]. In contrast, FIG. 16e shows that unilateral infusions of βCCt into the CPu/NACC areas were completely ineffective in altering alcohol-maintained responding [F(5, 40)=0.466, p>0.05].

Locomotor Sedation Study: Interaction of βCCt and Chlordiazepoxide

P and HAD-1 Rats

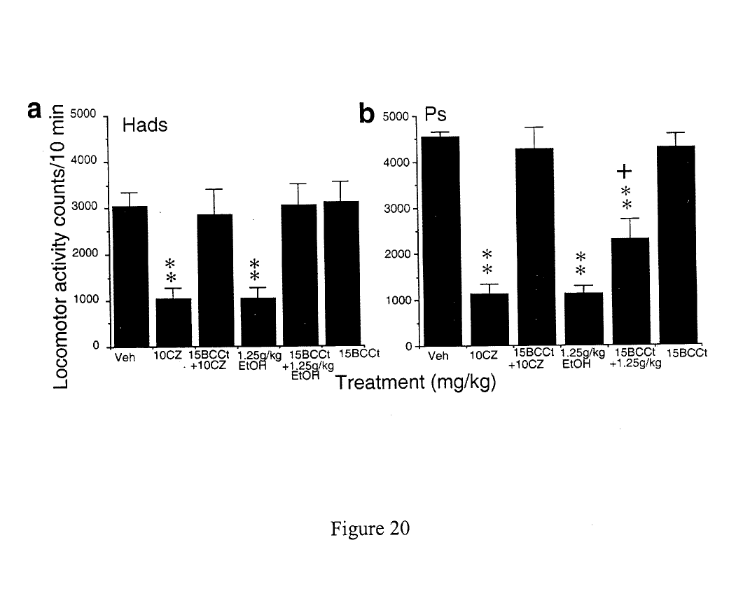

FIGS. 20a-b illustrate the sedative profile of chlordiazepoxide and EtOH. Chlordiazepoxide (10 mg/kg, i.p.) and EtOH (1.25 g/kg, i.p.) produced a profound and comparable reduction in locomotor activity compared with the vehicle treated controls in the P and HAD-1 rats. These findings resulted in a highly significant effect of drug treatment in P and HAD-1 rats [F(5, 36)=8.67, p<0.0001] and [F(5, 36)=30.99, p<0.0001], respectively. βCCt (15 mg/kg, i.p.) reversed the sedation produced by both chlordiazepoxide and EtOH in both P [(p<0.01), (p<0.01), respectively] and HAD-1 [(p<0.01) (p<0.01), respectively] rats. Given alone, βCCt did not produce any intrinsic effects in either rat line (p>0.05).

Discussion

Parenternal administration of βCCt selectively reduced alcohol-maintained responding but did not alter responding maintained by a highly palatable reinforcer in P rats. In HAD-1 rats, βCCt continued to exhibit selectivity in suppressing alcohol-maintained responding compared with a caloric reward. Further, a four-fold higher dose of βCCt was required to reduce consumption of the sucrose reinforcer compared to alcohol in this line. Previous research has suggested that when response rates for a drug and alternative reinforcer approximate each other at basal levels, rate is no longer a confounding factor contributing to the effects of an antagonist in drug self-administration studies (Samson et al., 1989; Carroll et al., 1989; Petry and Heyman, 1995; June in press). Response rates for EtOH and the alternative reinforcers were similar in P and HAD-I rats. Hence, βCCt evidenced a marked specificity in reducing alcohol responding compared with responding maintained by other palatable ingesta across two alcohol-preferring lines.

Consistent with the effects observed following parenternal administration, direct VP infusions of βCCt produced dose-dependent reductions in alcohol-maintained responding in both P and HAD-1 rats, but failed to alter responding for a palatable saccharin reward in P rats. These data reinforce the notion that the βCCt-induced reduction on alcohol-maintained behaviors was not due to a general suppression of consummatory behaviors. The βCCt-mediated, suppression also exhibited neuroanatomical specificity in P and HAD-1 rats. Thus, suppression was seen at the anterior to posterior VP levels in P and HAD-1 rats, but was not observed with the more dorsal placements in the NACC or CPu in either rat line. Further, this selective topography could clearly be demonstrated even following occupancy of the GABA_{A1} receptors by βCCt in a single hemisphere. The failure of βCCt to alter alcohol self-administration in the NACC and the CPu are consistent with previous reports of a lack of _{A1} transcript in the NACC and CPu (Churchill et al., 1991; Araki and Tohyama, 1991; Turner et al., 1993; Fritchy and Mohler, 1995; Duncan et al., 1995). These data are also consistent with the marginal levels of [³H] zolpidem binding (a GABA_{A1} selective agonist) in the NACC and CPu (Duncan et al., 1995). Criswell and his colleagues (1993;1995; Duncan et al., 1995) have suggested zolpidem binding sites are predictive of loci where EtOH potentiates GABAergic function in the CNS.

The VP has been reported to play a role in regulating the rewarding properties of both psychostimulant and opioid drugs (Austin and Kalivas, 1990; Hubner and Koob, 1990; Hliroi and White, 1993; Gong et al., 1996;1997; Johnson and Napier, 1997). However, there has been no direct link of this substrate to the rewarding properties of EtOH. The present study and work with 3-PBC, another GABA_{A1} antagonist (Carroll et al., 2000; Harvey et al., in press), represent the first direct test of the hypothesis. However, it is not known if the GABA_{A1} receptors of the VP are sufficient to regulate alcohol's reinforcing properties. The VP has a small, albeit much lower density of non-_{A1} containing GABA receptors (Turner et al., 1993; Fritschy and Mohler, 1995; Wisden et al., 1992). Moreover, the selectivity of βCCt at the GABA_{A1} receptor compared with the _{A2} and _{A3} subtypes is also important to note. Recombinant receptor studies show βCCt exhibits a >10 fold selectivity for the GABA_{A1} over the _{A2} and _{A3} receptors, and a >110 fold selectivity for the _{A1} over the _{A5} subtype (Cox et al., 1995). Thus, binding of βCCt at non-_{A1} receptors might contribute to the reduction in alcohol responding, even following direct infusion in the VP. However, this hypothesis is mitigated by the failure of βCCt to alter alcohol responding in the NACC and CPu, where greater levels of _{A2} and _{A3} transcripts have been observed (Turner et al., 1993; Fritschy and Mohler, 1995; Wisden et al., 1992). In addition, GABAergic involvement within the mesolimbic DA or opioid systems in the VP also cannot be ruled out (Austin and Kalivas, 1990; Kalivas et al., 1993a). Taken together, the present data do not unequivocally support the role for the GABA_{A1} receptor as the sole mediator of the antagonistic actions of βCCt, but is the most tenable explanation of the neuromechanisms by which βCCt selectively reduces alcohol responding.

Previous research has demonstrated that parentemal and oral administration of the pyrazoloquinoline (CGS 8216) and the β-carboline (ZK 93426) antagonists can selectively reduce alcohol-maintained responding in P rats (June et al., 1998b). Twenty-four hours post-drug administration, the suppression was still detectable with higher doses (≧20 mg/kg). In contrast, the antagonist flumazenil did not alter EtOH-maintained responding. A single infusion directly into the VP or systemic injection of βCCt was also observed to selectively suppress alcohol responding 24 hr post-drug administration under some conditions in P (FIG. 13a) and HAD-1 rats (FIG. 16d). βCCt's long duration of action makes this compound ideal as a prototype pharmacotherapeutic agent for use with humans. Its longevity in vivo can be attributable to its 3-carboxylate t-butyl ester configuration, which cannot be readily hydrolyzed by esterase activity. T-butyl ester ligands are apparently too large to fit in the esterase active site and consequently are longer lived in vivo (Zhang et al., 1995).

The locomotor sedation produced by chlordiazepoxide was reversed by βCCt (15 mg/kg, i.p.) in HAD-1 and P rats. These data are consistent with previous research in mutant mice suggesting that the GABA_{A1} receptor subtype mediates the sedative actions of BDZs (Rudolph et al., 1999; McKeman et al., 2000). However, the current study extends these findings by demonstrating that the GABA_{A1} receptor may also play a significant role in regulating the sedation produced by an intoxicating dose of alcohol (1.25 g/kg). These data are also consistent with our previous research demonstrating that ZK 93426 and CGS 8216, but not flumazenil are capable of blocking the sedative actions of alcohol (June et al., 1998e). Hence, given that ZK 93426 and CGS 8216 are nonselective antagonists, it is possible that the βCCt reversal/attenuation of the alcohol-induced sedation may be regulated in part, by other non-_{A1} receptor subtypes. Nevertheless, when given alone, βCCt did not produce intrinsic effects on motor activity in either rat line. These data are in agreement with the work of Griebel et al., (1999) who reported that doses as high as 60 mg/kg failed to produce intrinsic activity in the open field in mice. Together, the above studies suggest that βCCt is devoid of intrinsic effects on locomotor behaviors and its antagonism of the sedation produced by chlordiazepoxide and alcohol may be mediated via the GABA_{A1} receptor subtype. Thus, βCCt may be used as a pharmacological tool for distinction among the GABA_{A} receptor subtypes for selected behaviors of alcohol, as well as BDZs.

Based on the present findings, we contend that in contrast to previous reports (for review see Jackson and Nutt, 1995), a large negative intrinsic efficacy is not a prerequisite for BDZ ligands to antagonize the rewarding or sedative properties of alcohol. Thus, subtype selectivity, as well as efficacy, may be a more important predictor of a BDZ ligand's capacity to selectively antagonize alcohol's neurobehavioral actions in the absence of intrinsic effects (June et al., 2001). In further support of this hypothesis, molecular modeling studies have shown that the pharmacophore for high affinity and selectivity at the GABA_{A1} receptor is clearly different for βCCt compared from the prototypical BDZ antagonist flumazenil (Cox et al., 1998).

In conclusion, the present study demonstrated that in two rodent models of chronic alcohol consumption (e.g., P and HAD-1 lines), βCCt produces reliable and selective antagonism of EtOH-seeking behaviors. We further demonstrated that the βCCt suppression was regulated in part, via the VP, a mesolimbic substrate purported to play a role in the reinforcing properties of other abuse drugs (Hubner and Koob, 1990; Gong et al., 1996; Johnson and Napier, 1997). βCCt's selectivity at the GABA_{A1} receptor and low intrinsic efficacy (close to GABA neutral) across the various receptor subtypes may contribute to its failure to exhibit locomotor-impairing effects in the current study. It has been suggested that greater side effects of BDZs occur with ligands that lack receptor subtype selectivity (Stephens et al., 1992). However, the degree to which the oocyte data accurately reflects βCCt's in vivo action remains to be determined. Nevertheless, we conclude that βCCt may have potential as a prototype pharmacotherapeutic agent to effectively reduce alcohol drinking behavior in human alcoholics. βCCt's capacity to reduce alcohol's euphorigenic properties while concurrently eliminating or attenuating its motor-impairing effects should render it an optimal prototype in the development of pharmacotherapeutic agents to treat alcohol dependent individuals.

### EXAMPLE III

### Anti-Axiety Property

Because anxiety has been suggested to play a role in some forms of alcohol abuse in humans (Schuckit, 1987; Kushner et al., 1990; Kessler et al., 1997), we hypothesized that if an agent was capable of reducing alcohol-seeking behaviors, while concomitantly relieving anxiety, it might function as an ideal pharmacotherapy in alcohol-dependent individuals, particularly those diagnosed with an anxiety-related disorders (i.e., phobias, generalized anxiety, obsessive compulsions). Previous research has demonstrated that some BDZ antagonists were capable of displaying unusual anxiolytic actions (Jensen et al., 1984; De Vry and Slangen, 1985; Pellow and File, 1986; Gonzalez and File, 1997). In this study, we assessed the functional capacity of βCCt to competitively antagonize the anxiolytic versus the sedative properties of the prototypical BDZ, chlordiazepoxide. We also employed human HEK cells to determine if βCCt exhibits effects most similar to a BDZ agonist (e.g., chlordiazepoxide) or an antagonist (e.g., flumazenil).

Material and Methods

Dose-response analysis of βCCt

Male P rats (N=40) from the S48 and S49 generations and female HAD-1 rats (N=37) from the S34 generation were used. Rats were approximately 3 months of age and weighed between 247 and 325 g at the beginning of the experiment.

Anxiolytic and Locomotor Sedation Studies

Elevated plus Maze

Rats were placed in the middle of the automated plus maze (Acuscan Electronics, Columbus, Ohio, USA) with two walled and open arms under dim illumination (for details see June et al., 1998b). Changes in the percentage of time spent on the open arms indicate changes in anxiety (Pellow et al., 1985). Because the number of closed arm entries has been suggested to be the best measure of locomotor activity (Belzung et al., 1994; Cruz et al., 1994; File, 1995), and it is possible that a pharmacological agents might increase time spent on the open arm secondary to increases in locomotion (Belzung et al., 1994; Cruz et al., 1994), this parameter was also evaluated.

Locomotor Activity

Ambulatory count in the open field was recorded individually for 10 min in a plexiglas chamber (42 cm×42 cm 30 cm) using a Digiscan activity monitoring system (Acuscan Electronics, Columbus, Ohio, USA) (for details of the monitoring system see June et al., 1998b).

Systemic Injection Procedures

βCCt (5-60 mg/kg i.p.) and chlordiazepoxide (2.5, 5, 10 mg/kg i.p.) were administered 15 and 30 min, respectively, prior to being placed in the plus maze or open field. EtOH (1.25 g/kg) was given 5 min prior to placing the rats in the open field. When βCCt was given in combination with either chlordiazepoxide or EtOH, it was given 10 min prior to chlordiazepoxide and EtOH. As noted above, βCCt was administered in Tween-20 solution, while all other drugs were mixed in sterile saline. Animals were tested between 9am-3 pm.

Statistical Analysis for Interactional Studies

HAD-1 and P rats were randomly assigned to each drug treatment group. A between group ANOVA with drug treatment as the independent factor was conducted for HAD-1 (n=6-10 per treatment group) [total N=48] and P (n=6-11 per treatment group) [total N=48] rats on the plus-maze test. A between group ANOVA with drug treatment (i.e., dose) as the independent factor was also conducted for HAD-1 (n=6-9 per treatment group) [total N=42] and P (n=6-8 per treatment group) [total N=42] rats on the locomotor activity parameter (i.e., ambulatory count). Post-hoc comparisons between individual drug treatment groups were made using the Newman Keuls Test in all experiments.

Statistical Analysis for μCCt Dose-response Analyses

HAD-1 asnd P rats were randomly assigned to each of the four drug treatment groups (Veh, 5, 15, 30, 60 mg/kg). A between group ANOVA with drug treatment as the independent factor was conducted for HAD-1 (n=8-12 per treatment group) [total N=42] and P (n=7-12 per treatment group) [total N=42] rats on the plus-maze test.

Results

Anti-anxiety Effects of 1CCt

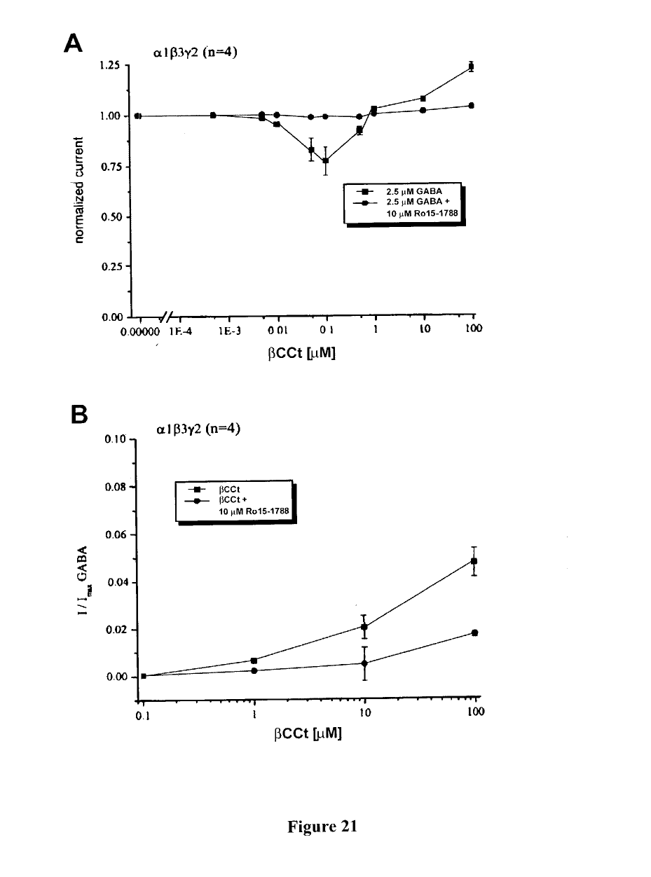

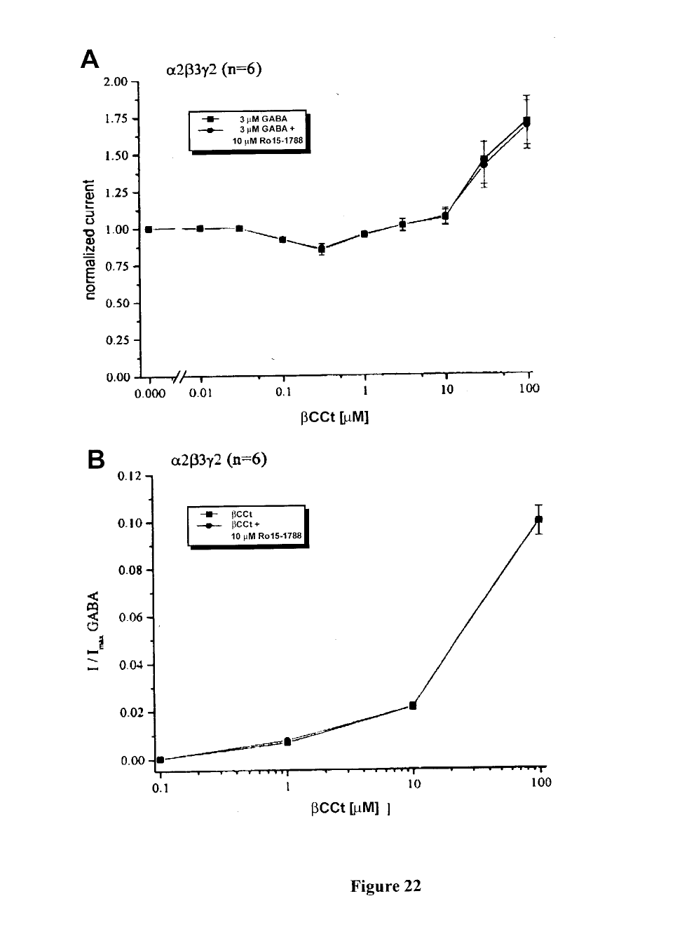

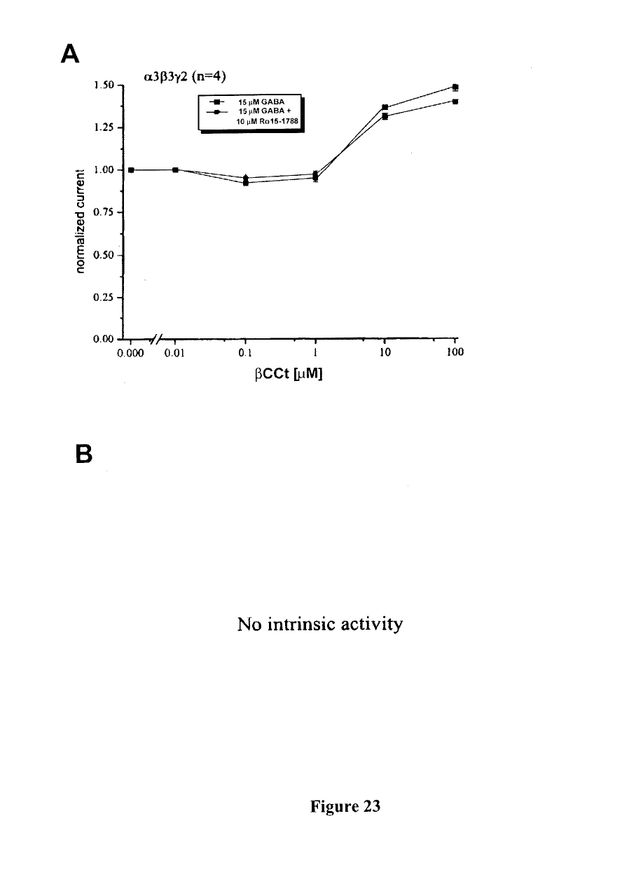

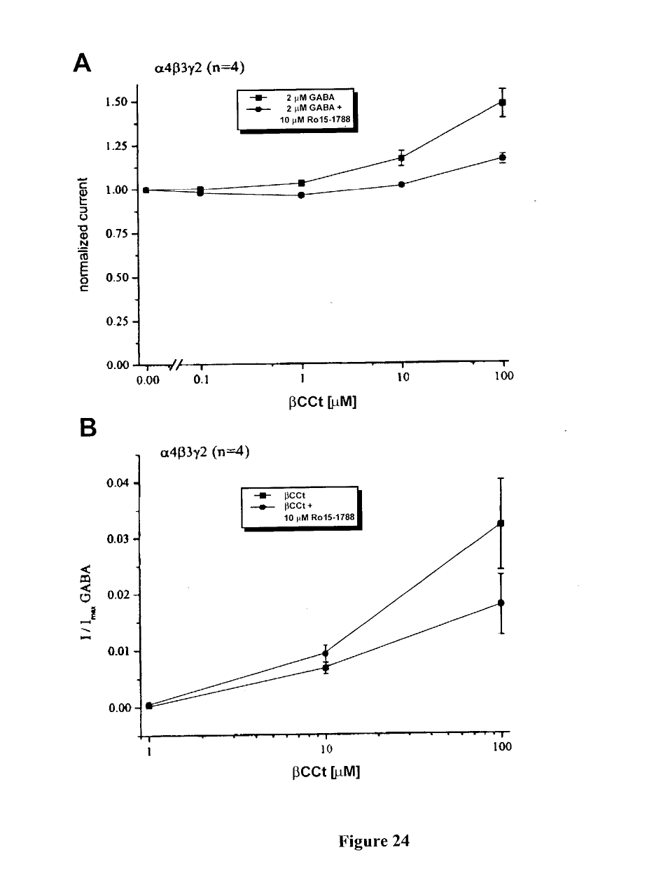

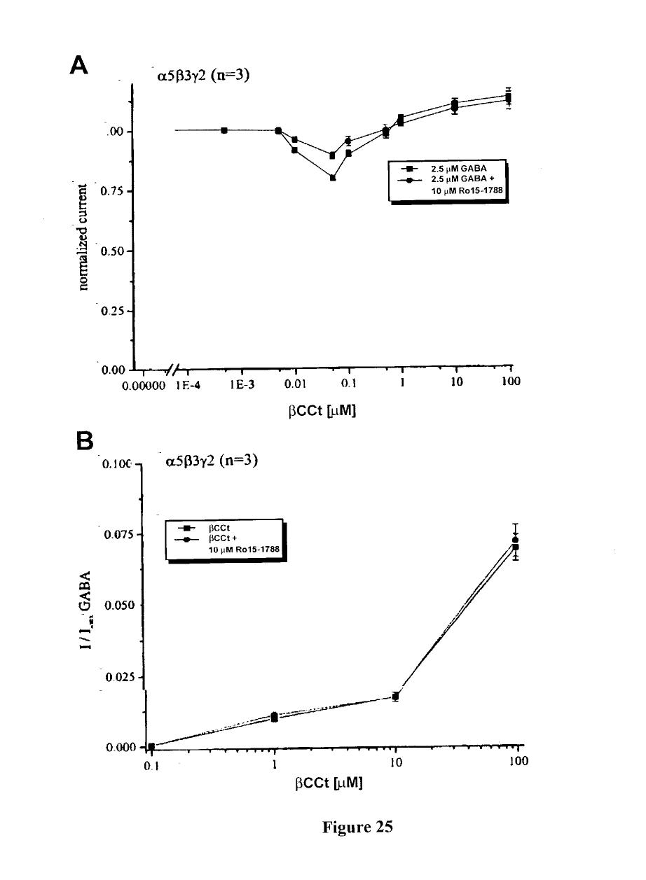

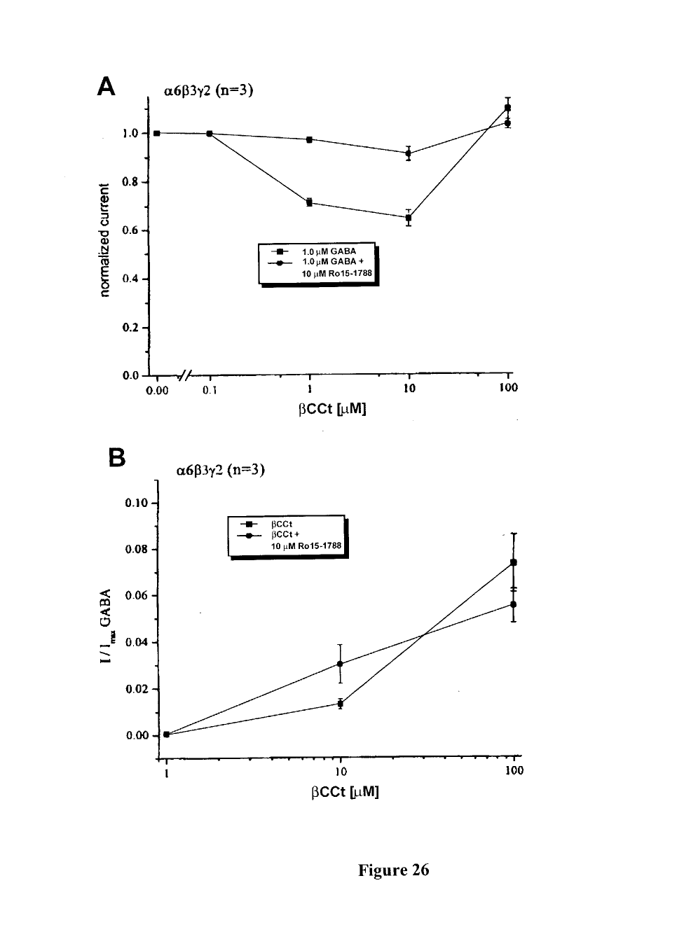

FIGS. 21-26 represent the molecular biology data in α1-α6 GABA_{A} receptors. Unlike the molecular biology data in Xenopus oocytes, human HEK cells are employed in this study to determine if βCCt exhibits effects most similar to a BDZ agonist (e.g., chlordiazepoxide) or an antagonist (e.g., flumazenil). The most salient findings of these data is that, at the α2 and α3 receptors, βCCt displays effects very similar to that of an agonist. These findings are particularly interesting in light of the previous data in the literature on α2 knockout mice demonstrating that it is the α2 GABA receptor that plays a role in regulating anxiety. In other words, βCCt behaves like chlordiazepoxide at the anxiety receptors. It is important to note that these effects occur at 50-100 μM, and would most likely be observed at moderate doses.

Anxiolytic Study: Dose-response Analyses of βCCt

P and HAD-1 Rats

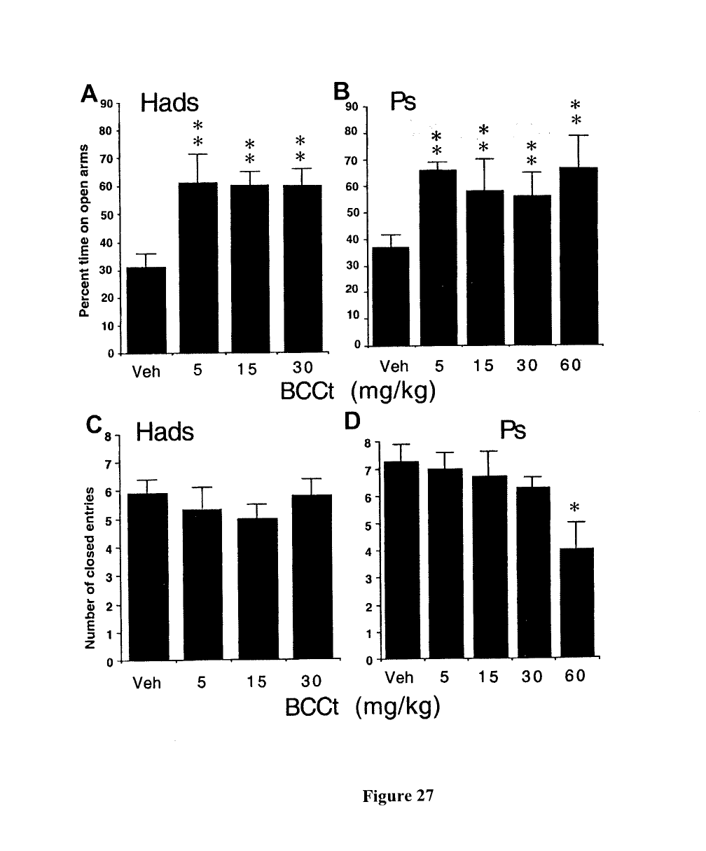

Dose-response analyses for βCCt (5-60 mg/kg, i.p.) were performed in new samples of HAD-1 and P rats. All βCCt doses produced marked increases in the percentage of time spent on the open arms in the HAD-1 [F(3, 34)=7.72, p<0.0005] and P [F(4, 34)=4.98, p<0.0029] rats relative to their respective vehicle control groups. However, no dose effect was observed in either rat line (FIGS. 27A-27D). Specifically, in the HAD-1 line, a dose of 5 mg/kg was effective as a 30 mg/kg dose (p>0.05), while in the P line, a 5 mg/kg dose was effective as the 60 mg/kg dose (p>0.05). In the HAD-1 rats, the 60 mg/kg dose produced a marked sedative profile in the plus maze, precluding evaluating of βCCt's anxiolytic activity (data not shown). In contrast to the anxiolytic effects, βCCt was without effect on the number of closed arm entries in the HAD-1 rats [F(4, 34)=0.187, p>0.05], and produced a significant reduction in performance only with the 60 mg/kg dose in the P rats [F(4, 34)=2.58, p<0.05]. Thus, similar to the open-field parameters, βCCt was essentially devoid of locomotor sedation in the plus maze, except at very high doses.

Anxiolytic Study: Interaction of βCCt and Chlordiazepoxide

P and HAD-1 Rats

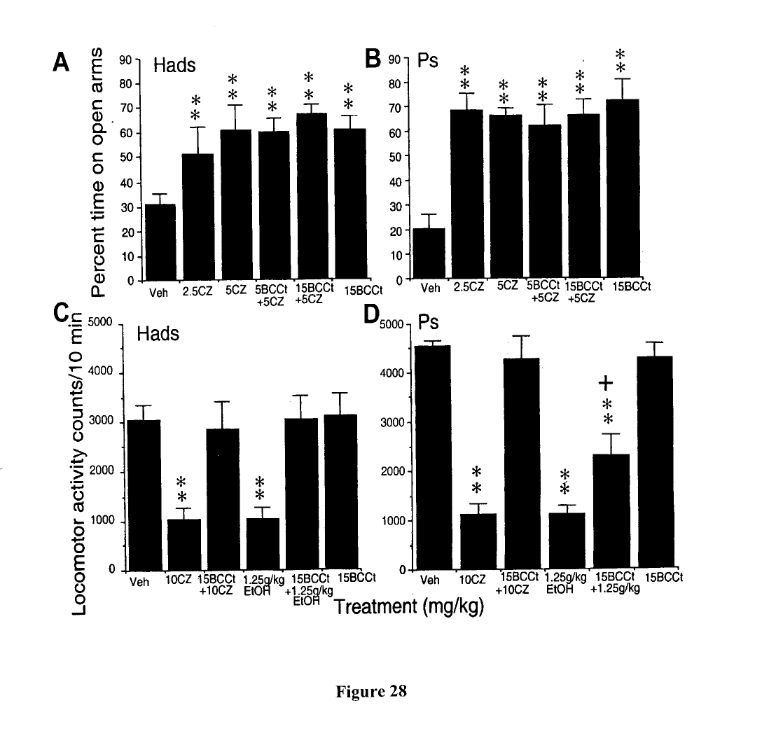

Chlordiazepoxide (2.5, 5 mg/kg, i.p.) and βCCt (15 mg/kg, i.p.) produced marked, and comparable anxiolytic-like activity in both HAD-1 and P rats as evidenced by an increase in the percentage of time spent on the open arms in the plus maze [F(5, 43)=10.58, p<0.0001], [F(5, 42)=4.37, p<0.0027], respectively. These findings were confirmed by post-hoc analyses [p≦0.01]. However, neither dose of βCCt (5, 15 mg/kg, i.p.) altered the anxiolytic activity produced by chlordiazepoxide (5.0 mg/kg, i.p.) in HAD-1 [p>0.05, p>0.05] or P [p>0.05, p>0.05] rats (FIGS. 28A and 28B).

Locomotor Sedation Study: Interaction of βCCt and Chlordiazepoxide

P and HAD-1 Rats

The sedative profile of chlordiazepoxide and EtOH has been previously described. Chlordiazepoxide (10 mg/kg, i.p.) and EtOH (1.25 g/kg, i.p.) produced a profound and comparable reduction in locomotor activity compared with the vehicle treated controls in the HAD-1 and P rats resulting in a highly significant effect of drug treatment [F(5, 36)=30.99, p<0.0001], [F(5, 36)=8.67, p<0.0001], respectively. In contrast to the anxiolytic effects, the sedation produced by both chlordiazepoxide and EtOH was reversed in a competitive manner by βCCt (15 mg/kg, i.p.) in HAD-1 [(p<0.01), (p<0.01), respectively] and P [(p<0.01) (p<0.01), respectively] rats. Given alone, βCCt (15 mg/kg, i.p.) did not produce any intrinsic effects (p>0.05) (FIGS. 28C and 28D).

Discussion

βCCt Exhibits Anxiolytic Activity in P and HAD-1 Rats

βCCt was ineffective in blocking the anxiolytic activity produced by chlordiazepoxide in HAD-1 or P rats in the plus maze test, even with doses as high as 60 mg/kg (data not shown). However, given alone, βCCt produced marked, and comparable anxiolytic effects as did chlordiazepoxide. As a result, complete dose-response analyses were conducted in both rat lines. In P rats, a dose range of 5-60 mg/kg produced anxiolytic effects, while a 5-30 mg/kg dose range was effective in the HAD-1 rats. The anxioselective effects of βCCt were clearly evident in both lines, as locomotor activity was reduced only with the highest dose of 60 mg/kg. Interestingly, however, the reduction in the number of close arm entries did not preclude the 60 mg/kg dose from producing anxiolytic activity in the P rat. However, in the HAD-1 rats, the 60 mg/kg dose was sedative and precluded evaluation of anxiolytic activity (data not shown). While βCCt was anxioselective in both lines, it failed to exhibit a dose response profile across a rather wide dose range. The rationale for this is not known at present, but it may reflect a lower distribution of GABA-BDZ receptors in alcohol-preferring rats compared with their low alcohol drinking counterparts (i.e., NP, LAD-1). Some support for this hypothesis is evidenced by the fact that compared with the P and HAD-1 rats, NP and LAD-1 rats exhibit a marked reduction in sensitivity to the anxiolytic actions of some BDZ ligands (Carroll et al., 2001). Thus, low levels of BDZ receptors could become maximally occupied even with doses as low as 5 mg/kg in alcohol-preferring rat lines. Taken together, the above data are consistent with previous research showing that the α₁₋subtype does not mediate the anxiolytic actions of BDZs (Rudolph et al., 1999; McKernan et al., 2000); however, they contrast other reports implicating the α₁₋subtype in the anxiolytic actions of BDZs (Belzung et al., 2000; Rowlett et al., 2001). These differences could be due to the type of species, or the anxiolytic paradigms used in the prior studies. Similar to the present study, the Rudolph et al., (1999) and the McKernan et al., (2000) employed the plus maze as the principal anxiolytic measure. Moreover, our study demonstrated that βCCt exhibits similar agonist effects to anxiety receptors, α₂ subtype receptors, like chlordiazepoxide. Therefore, we hypothesized that the anxiolytic effects produced by βCCt were mediated by non-α₁₋subtype receptors.

βCCt Antagonizes the Sedation of Chlordiazepoxide and Alcohol in P and HAD-1 Rats

In contrast to the anxiolytic data, the locomotor sedation produced by chlordiazepoxide and the intoxicating dose of EtOH (1.25 g/kg) was completely antagonized/attenuated in a competitive manner by βCCt (15 mg/kg, i.p.) in HAD-1 and P rats. Thus, these data are consistent with α₁₋subtype mediation of the sedative actions of BDZs (Rudolph et al., 1999; McKernan et al., 2000) as well as alcohol. These data are also consistent with our previous research demonstrating that “some” BDZ antagonists (e.g., ZK 93426 and CGS 8216) are capable of blocking the sedative effects of alcohol (June et al., 1998c). Given alone, βCCt did not produce intrinsic effects in either rat line. These data are consistent with the inability of βCCt to alter the locomotor parameter in the plus maze test. They are also in agreement with the work of Griebel et al., (1999) who reported that doses as high as 60 mg/kg failed to produce intrinsic activity in the open field in mice. Taken together, the above studies suggest that βCCt is a safe, nontoxic BDZ antagonist, devoid of adverse effects whose actions may be mediated via the α1 receptor subtype for some, but not all of its neurobehavioral effects. Thus, βCCt may be used as a pharmacological tool for distinction among the GABA BDZ receptor subtypes for selected behaviors of BDZs, as well as alcohol.

### EXAMPLE IV

### Selective Suppressant Effects for Alcohol

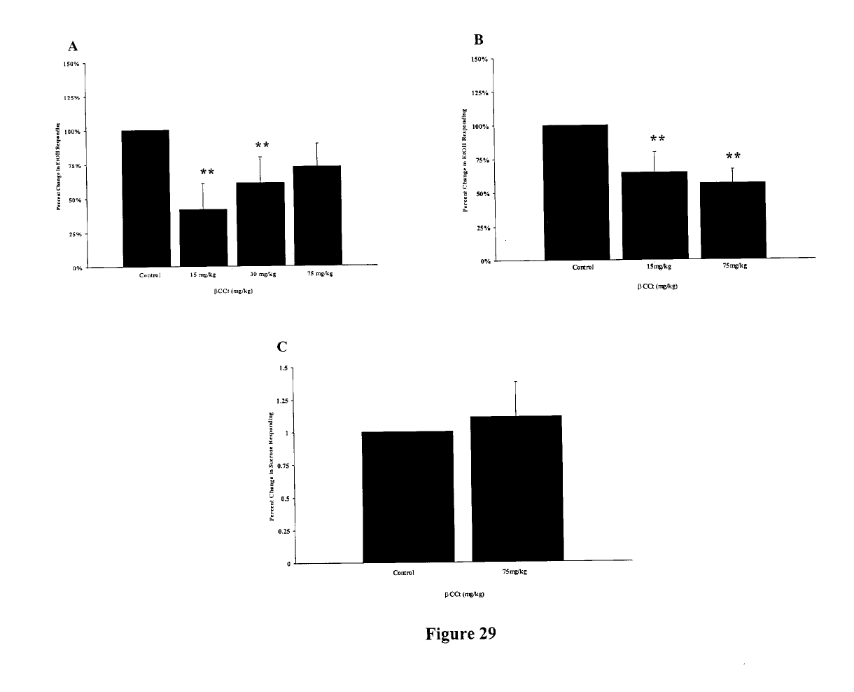

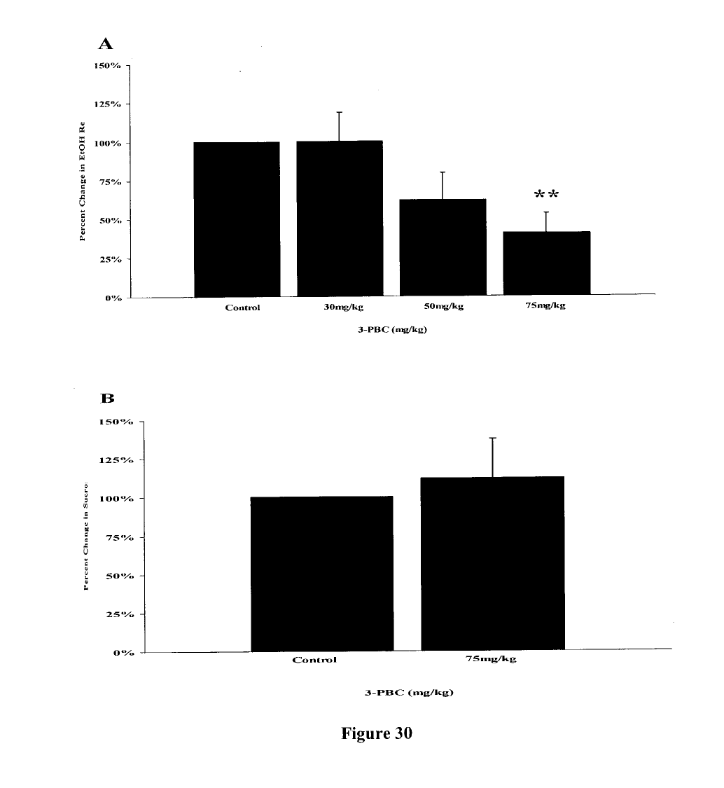

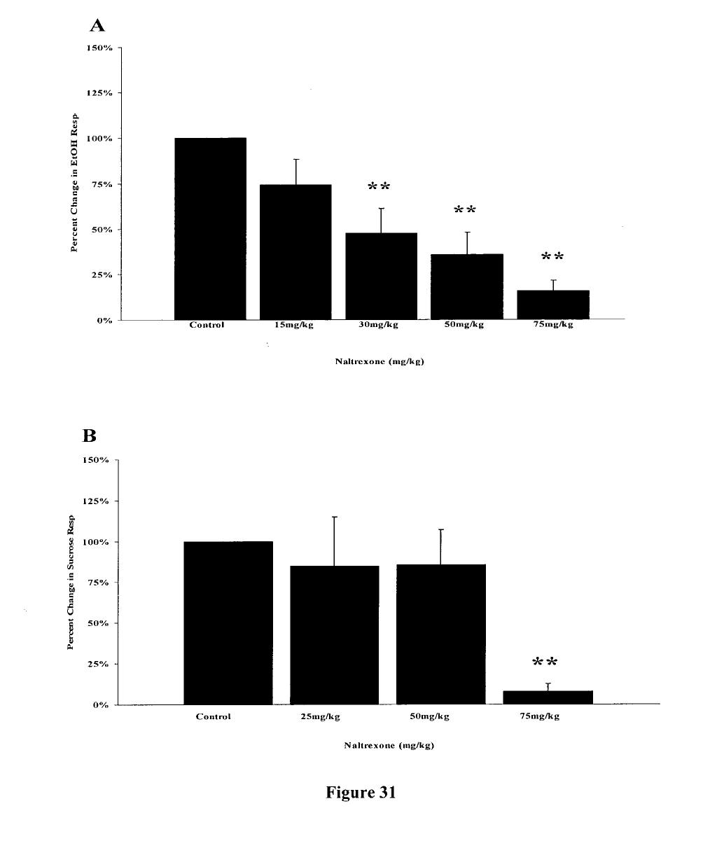

In this study we compared the suppressant effect of βCCt and 3-PBC with naltrexone, a currently used alcohol antagonist on the market. As it is shown in FIG. 29A-B, orally administered βCCt can reduce alcohol responding, however, the effects are not dose related. In addition, 24 hr after administration of βCCt, it continues to reduce alcohol responding with selected doses. Unlike βCCt, as shown in FIG. 30A, the effects of 3-PBC in reducing alcohol responding are dose related and 24 hr after administration, the suppressant effects of 3-PBC had dissipated. The suppressant effects of naltrexone in reducing alcohol responding are also dissipated 24 hr after the administration. Additionally, it is demonstrated that the highest does of βCCt (75 mg/kg) and 3-PBC (75 mg/kg) completely fail to alter responding for a control sucrose solution (FIGS. 29C and 30B), while the highest dose of naltrexone produced a profound suppressant effect on responding for a control sucrose solution (FIG. 31B). This suggests that the suppressant effects observed by naltrexone on alcohol responding with high doses are not selective for alcohol. Unlike naltrexone, the suppressant effects observed by βCCt and 3-PBC are selective for alcohol, and not reinforcers in general.

### REFERENCES

Araki T, Tohyama M (1991): Region-specific expression of GABA_{A} receptor α3 and α4 subunits mRNAs in the rat brain. Mol Brain Res 12:295-314.

Austin M C, Kalivas P W (1990): Enkephallinergic and GABAergic modulation of motor activity in the ventral pallidum. J Pharmacol Exper Ther 252:1370-1377.

Barnard E A, Skolnick P, Olsen R W, Möhler W, Sieghart W, Biggio G, Braestrup C, Bateson A N, Langer S Z (1998): International union of pharmacology. XV. Subtypes of γ-aminobutyric acid receptors: Classification on the basic of subunit structure and receptor function. Pharmacol Rev 50: 291-313.

Barrett J E, Brady L S and Witkin J M (1985) Behavioral studies with anxiolytic drugs I. Interaction of the benzodiazepine antagonist RO15-1788 with chlordiazepoxide, pentobarbital and ethanol. J Pharmacol Exp Ther 233:554-559.

Carroll M E, Lac S T, Nygaard S L (1989): A concurrently available nondrug reinforcer prevents the acquisition or decreases the maintenance of cocaine-reinforced behaviors. Psychopharmacology 97:23-29.

Carroll M, Foster K, Harvey S, Mc Kay P F, Cook J M, June H L (2000): Selective GABA_{A}-α1 subunit ligands (BCCt, 3-PBC) attenuate responding maintained by ethanol following microinjection into the ventral pallidum. Alcoholism: Clin Exp Res 24:47A [Abstract #24].

Carroll M, Woods J E II, Seyoum R A, June H L (2001): The role of the GABAa α1 subunit in mediating the sedative and anxiolytic properties of benzodiazepines. Alcoholism: Clin Exper Res 25: 12A.

Churchill L, Bourdelais A, Austin S, Lolait S J, Mahan L C, O'Carroll A M, Kalivas P W (1991): GABA_{A} receptors containing α1 and β2 subunits are mainly localized on neurons in the ventral pallidum. Synapes 8:75-85.

Churchill L, Kalivas P W (1994): A topographical organized GABA projection from the ventral pallidum to the nucleus accumbens in the rat. J Comp Neurol 345:579-595.

Cicero T J, A critique of animal analogues of alcoholism (1979) in Biochemistry and pharmacology of ethanol, Vol. 2, (Majchrowicz E and Noble E P, eds), pp 533-560. New York: Plenum Press.

Cloninger (1987): Neurogenetic adaptive mechanisms in alcoholism. Science 236: 410-416.

Cox E D, Hagen T J, McKernan R M, Cook J M (1995): BZ1 receptor specific ligands: Synthesis and biological properties of BCCt, a BZ1 receptor subtype specific antagonist. Med Chem Res 5:710-718.

Cox E D, Diaz-Arauzo H, Huang Q, Reddy M S, Ma C, Harris B, McKernan R M, Skolnick P, Cook J M (1998): Synthesis and evaluation of analogues of the partial agonist 6-Propyloxy)-4-(methoxymethyl)-β-carboline-3-carboxylic acid ethyl ester (6-PBC) and the full agonist 6-(Benzyloxy)-4-(methoxymethyl)-β-carboline-3-carboxylic acid ethyl ester (ZK 93423) at wild type and recombinant GABA_{A} receptors. J Med Chem 41:2537-2552.

Criswell H E, Simson P E, Duncan G E, Mc Cown T J, Herbert J S, Morrow L, Breese G R (1993): Molecular basis for regionally specific action of ethanol on γ-aminobutyric acid_{A} receptors: Generalization to other ligand-gated ion channels. J Pharmacol Exper Ther 267:522-527.

Criswell H E, Simson P E, Knapp D J, Devaud L L, Mc Cown, T J, Duncan G E, Morrow A L, Breese G R (1995): Effect of zolpidem on γ-aminobutyric acid (GABA)-induced inhibition predicts the interaction of ethanol with GABA on individual neurons in several rat brain regions. J Pharmacol Exper Ther 273:525-536.

Duncan G E, Breese G R, Criswell H E, McCown T J, Herbert J S, Devaud L L, Morrow A L (1995): Distribution of {³1H}zolpidem binding sites in relation to messenger RNA encoding the α1, β2 and γ2 Subunits of GABA_{A} receptors in rat brain. Neuroscience 64:1113-1128.

File S E, Baldwin H A, Hitchcott P K (1989): Flumazenil but not nitrendipine reverses the increased anxiety during ethanol withdrawal in the rat. Psychopharmacology 98:262-264.

File S E, Pellow S (1986): Intrinsic actions of the benzodiazepine receptor antagonist RO 15-1788. Psychopharmacology 88: 1-11.

Fritschy J M, Mohler H (1995): GABA_{A}-receptor heterogenetity in the adult rat brain. Differential regional and cellular distribution of seven major subunits. J Comp Neurol 359:154-194.

Gong W, Neill D, Justice J B (1996): Place preference conditioning and locomotor activation induced by local injection of psychostimulants into ventral pallidum. Brain Res 707:64-74.

Gong W, Justice J B, Neill D (1997): Dissociation of locomotor and conditioned place preference responses following manipulation of GABA-A and AMPA receptors in ventral pallidum. Prog Neuropsychophannacol Bio Psych 21: 839-852.

Griebel G, Perrault G, Letang V, Grainger P, Avenet P, Schoemaker H and Sanger D J (1999): New Evidence that the pharmacological effects of benzodiazepine receptor ligands can be associated with activities at different BZ (α) receptor subtypes. Psychopharmacology (Berlin) 146:205-213.

Groenewegen H J, Vermeulen-Van Der Zee E, Te Kortschot A, Witter M P (1987) Organization of the projections to the ventral striatum in the rat. A study using anterograde transport of phaseolus vulgaris leucoagglutinin. Neuroscience 23: 103-120.

Groenewegen H J, Berende H W Haber S N (1993): Organization of the output of the ventral striatopallidal system in the rat: Ventral pallidal efferents. Neuroscience 57:113-142.

Haefely W (1983): Antagonists of benzodiazepine: functional aspects. In: benzodiazepine recognition. Site Ligands: Biochemistry and Pharmacology. (Biggio G, Costa E, eds), p. 73. New York: Raven.

Haefely W (1985) Pharmacology of benzodiazepine antagonists. Pharmacopsychiatry. 18:163.

Harris C M, Lal H (1988): Central nervous system effects of Ro15-4513. Drug Dev Res 13:187-203

Harvey S C, McIntosh J M, Cartier G E, Maddox F N (1997): Determinants of alpha-conotoxin MII on α3β2 neuronal nicotinic receptors. Molec Pharmacol 51:336-342.

Harvey S C, Foster K L, McKay P F, Carroll M, Seyoum R, Woods, James E. II, Grey C, McCane S, Cummings R, Mason D, Jones C M, Ma C, Cook J M, June, H L (in press). The GABA_{A} receptor α1 subtype in the ventral pallidum regulates EtOH-seeking behaviors. J Neurosci

Heimer L, Zahm D S, Churchill P, Kalivas W and Wohltmann C. (1991): Specificity in the projection patterns of accumbal core and shell in the rat. Neuroscience 41: 89-125

Heimer L, Alheid G (1991): Piecing together the puzzle of basal forebrain anatomy. In Napier, T C, Kalivas P W, Hanin I (eds), The basal forebrain: anatomy to function. New York, Plenum Press, pp 1-42.

Higgs S, Cooper S J (1995): Benzodiazepine receptor inverse agonists and ingestive behaviors: the palatability hypothesis. In: Benzodiazepine Receptor Inverse Agonists. (Sarter M, Nutt D J, Lister R G eds), pp. 163-184. New York: Wiley-Liss.

Hiroi N, White N M (1993): Place preference conditioning and locomotor activation induced by local injection of psychostimulants into the ventral pallidum. Brain Res 707:64-74.

Hiroi N, White N M (1993): The ventral pallidum area is involved in the acquisition but not the expression of the amphetamine conditioned place preference. Neuroscience Lett 156:9-12.

Huang Q, He X, Ma C, Liu R, Yu S, Dayer C A, Wenger G R, McKernan R, Cook J M (2000): Pharmacophore/receptor models for GABA_{A}/Bzr subtypes (α1β3γ2, α5β3γ2, and α6β3γ2) via a comprehensive ligand-mapping approach. J Med Chem 43: 71-95.

Hubner C B, Koob G F (1990): The ventral pallidum plays a role in mediating cocaine and heroin self-administration in the rat. Brain Res 508:20-29.

Hyytiä P, Koob G F (1995): GABA_{A} receptor antagonism in the extended amygdala decreases ethanol self-administration in rats. Eur Jn of Pharmacol 283:151-159.

Jackson H C, Nutt D J (1995): Inverse agonist and alcohol. In: Benzodiazepine Receptor Inverse Agonists. (Sarter M, Nutt D J, Lister R G eds), pp. 243-270. New York: Wiley-Liss.

Jensen L H, Petersen E N, Braestrup C, Honore T, Kehr W, Stephens D N, Schneider H, Seidelmann D, Schmiechen R (1984): Evaluation of the beta-carboline ZK93426 as a benzodiazepine receptor antagonist. Psychopharmacology 83:249-256.

Johnson P I, Napier T C (1997): Morphine modulation of GABA and glutamate-induced changes of ventral pallidal neuronal activity. Neuroscience 77:187-197

Johnson B A, Ait-Daoud N (2000): Neuropharmacological treatments for alcoholism: scientific basis and clinical findings. Psychopharmacology (Berlin) 149:327-344

June H L, Torres L, Cason C R, Hwang B H, Braun M R, Murphy J M (1998a): The novel benzodiazepine inverse agonist RO19-4603 antagonizes ethanol motivated behaviors: neuropharmacological studies. Brain Research 784: 256-275.

June H L, Zucarelli D, Craig K S, DeLong J, Cason C R, Torres L, Murphy J M (1998b) High affinity benzodiazepine antagonists reduce responding maintained by EtOH presentation in ethanol-preferring (P) rats. Jn Pharmacol Exper Ther 284: 1006-1014.

June H L, Cason C R, Cheatham G, Ruiyan L, Gan T, Cook J M (1998c) GABA_{A} benzodiazepine receptors in the striatum are involved in the sedation produced by a moderate, but not an intoxicating ethanol dose in outbred Wistar rats. Brain Res 794: 103-118.

June H L, Dejaravu S L, Williams J, Cason C R, Eggers M W, Greene T L, Leviege T, Torres L, Braun M R, Murphy J M (1998d): GABAergic modulation of the behavioral actions of ethanol in alcohol-preferring (P) and nonpreferring (NP) rats. Eur J Pharmacol 342: 139-151.

June H L, Grey C, Warren-Reese C, Lawrence A, Thomas A, Cummings R, Williams L, McCane S L, Durr L F, Mason D, (1998e): The opioid receptor antagonist nalmefene reduces alcohol motivated behaviors: preclinical studies in alcohol preferring (P) and outbred Wistar rats. Alcoholism: Clin Exper Res 22: 2174-2185.

June, H L, McCane, S., Zink, R. W., Portoghese, P., Li, T.-K. and Froehlich, J. C. (1999): The δ 2 opioid receptor antagonist naltriben reduces alcohol-motivated behaviors. Psychopharmacology 147: 81-89.

June H L, Harvey S C, Foster K L, McKay P F, Cummings R, Garcia M, Mason D, Grey C, McCane S L, Williams L, Johnson T B, Xiaohui H, Rock S, Cook J M (2001): GABA_{A}-receptors containing α5 subunits in the CA1 and CA3 hippocampal fields regulate ethanol-motivated behaviors: An extended ethanol reward circuitry. J Neurosci 21:2166-2177.

June, H. L., (in press) “Preclinical Models to Evaluate Potential Pharmacotherapeutic Agents in Treating Alcoholism and Studying the Neuropharmacological Bases of Alcohol-Seeking Behaviors”, In “Current Protocols in Neuroscience” (edited by J Crawley, C Gerfen, R McKay, M Rogawski, D Sibley and P Skolnick). John Wiley & Sons, Inc., Publisher, N.Y., N.Y.

Kalivas P W, Churchill L, Klitenick M A (1993a): GABA and enkephalin projection from the nucleus accumbens and ventral pallidum to the ventral tegmental area. Neurosci 57:1047-1060.

Kalivas P W, Churchill L, Klitenick M A (1993b): The circuitry mediating the translation of motivational stimuli into adaptive motor responses. In: Limbic Motor Circuits and Neuropsychiatry (Kalivas P W, Barnes C D, eds), pp 237-287. Boca Raton: CRC Press.

Koob G F, Roberts A J, Shulties G, Parsons L H, Heyser C J, Hyytia P, Merlo-Pinch E, Weiss F (1998) Neurocircuitry targets in ethanol reward and dependence. Alcoholism: Clin Exper Res 22:3-9.

Koob G F (1999) The role of the striato pallidal and extended amygdala systems in drug addction. Ann NY Acad Sci 877:445-460

Kranzler H R (2000): Pharmacotherapy of alcoholism: Gaps in knowledge ad opportunities for research. Alcohol 35:537-547.

Kuo H and Chang H T (1992): Ventral-pallidostriatal pathway in the rat brain: A light electron microscopic study. J Comp Neurol 321:626-636.

Lankford M F, Roscoe, A K, Pennington, S N and Myers, R D (1991): Drinking of high concentrations of ethanol vs. palatable fluids in alcohol-preferring (P) rats: valid animal model of alcoholism. Alcohol 8: 293-299.

Lankford M F and Myers, R D (1994): Genetics of alcoholism: simultaneous presentation of a chocolate drink diminishes alcohol preference in high alcohol drinking rats. Pharmacol Biochem Behav 49: 417-425.

Li T-K, Crabb D W, Lumeng L (1991): Molecular and genetic approaches to understanding alcohol-seeking behavior. In Meyer R E, Koob G F, Lewis M J, Paul S P (eds), Neuropharmacology of ethanol. Boston: Birkhauser, pp 107-124.

Li T-K (2000): Pharmacogenetics of responses to alcohol and genes that influence alcohol drinking. J Stud Alcohol 61: 5-12

Löw K, Crestani F, Keist R, Benke D, Brünig I, Benson J A, Fritschy J M, Rülicke T, Bluethmann H, Möhler H & Rudolph U (2000): Molecular and neuronal substrate for the selective attenuation of anxiety. Science 290:131-134.

Lui R Y, Hu R J, Zhang P W, Skolnick P, Cook J M (1996): Synthesis and pharmacological properties of novel 8-substituted imidazobenzodiazepines: high affinity, selective probes for α5 containing GABA_{A} receptors. Jn Med Chem 39:1928-1934.

Lumeng L, Murphy J M, McBride W J, Li T-K (1995): Genetic influences on alcohol preference in animals. In: The Genetics of Alcoholism (Begleiter H, Kissin B, eds), pp. 165-201. New York: Oxford University Press.

McBride W J, Li T (1998): Animal models of alcoholism: Neurobiology of high alcohol-drinking behavior in rodents. Critical Reviews in Neurobiology 12(4):339-369.

McKernan R M, Rosahl T W, Reynolds D S, Sur C, Wafford K A, Atack J R, Farrar S, Myers J, Cook G, Ferris P, Garrett L, Bristow L, Marshall G. Macaulay A., Brown N, Howell O, Moore K W, Carling R W, Street L J, Castro J L, Rgan C I, Dawson G R, Whiting P J. (2000): Sedative but not anxiolytic properties of benzodiazepines are mediated by the GABA_{A} receptor α₁ subtype. Nature Neurosci 3(6):587-592.

McKinzie D L, Eha R, Murphy J M, McBride W J, Lumeng L, Li T-K(1996): Effects of taste aversion training on the acquisition of alcohol drinking in adolescent P and HAD rat lines. Alcoholism Clinical and Experimental Research 20: 682-687.

Meisch R A, Lemaire G A (1993): Drug self-administration. In: Methods in behavioral pharmacology. (Van Haren F, ed), pp 257-300. New York: Elsevier.

Mogenson G J, Nielson M A (1983): Evidence that an accumbens to subpallidal GABAergic projection contributes to locomotor activity. Brain Res Bulletin 11: 309-314.

Napier T C, Chrobak J J (1992) Evaluation of ventral pallidal dopamine receptor activation in behaving rats. Neuroreport, 3:609-611.

Nauta H J, Smith, G P, Domesick V B, Faull R L M (1978a) efferent connections and nigral afferents of the nucleus accumbens septi in the rat. Neuroscience, 3:189-202.

Nauta W J H, Smith G P, Faull R L M and Domesick V B (1978b): Efferent connections and nigral afferents of the nucleus accumbens septi in the rat. Neuroscience 3: 385-401

Nowak K L, McBride, W J, Lumeng L, Li T-K, Murphy J M (1998): Blocking GABA_{A} receptors in the anterior ventral tegmental area attenuates ethanol intake of the alcohol-preferring P rat. Psychopharmacology 139:108-116.

O'Malley S S, Jaffe A J, Chang G, Schottenfeld R S, Meyer R E, Rounsaville B (1992): Naltrexone and coping skills therapy for alcohol dependence: A controlled study. Arch Gen Psychiatry 49: 881-887.

Paronis C A, Cox E D, Cook J M, Bergman J (2001): Different types of GABAa receptors may mediate the anticonflict and response rate-decreasing effects of zalepon, zolpidem, and midazolam in squirrel monkeys. Psychopharmacology (Berlin)156:461-468.

Paxinos G, Watson C (1998): The Rat Brain in Stereotaxic Coordinates. Sydney: Academic Press.

Petry, N. M., Heyman, G. M. (1995): Behavioral economic analysis of concurrent ethanol/sucrose and sucrose reinforcement in the rat: Effects of altering variable ratio requirements. J Exper Anal Behav 64:331-359.

Phillips A G, Fibiger H C (1991): Dopamine and motivated behavior: In sights provided by in vivo analyses. In P Willner and J Scheel-Kuger (eds), The Mesolimbic Dopamine System: From Motivation to Action. New York: John Wiley & Sons, pp 119-224.

Rowlett J K, Tornatzky W, Cook J M, Chunrong M, Miczek K A (2001): Zolpidem, triazolam, and diazepam decrease distress vocalizations in mouse pups: differential antagonism by flumazenil and the β-carboline-3-carbozylate-t-butyl ester (βCCt). J Pharmacol Exper Ther 297:247-253.

Rudolf U, Crestani F, Benke D, Brünig I, Benson J A, Fritschy J M, Martin J R, Bluethmann H, Mohler H (1999): Benzodiazepine actions mediated by specific γ-aminobutyric acidA receptor subtypes. Nature 401:796-800.

Sanger D J, Benavides J, Perrault G, Morel E, Cohen C, Joly D Zivkovic B (1994): Recent Developments in the behavioral pharmacology of benzodiazepine (omega) receptors: Evidence for the functional significance of receptor subtypes. Neurosci Biobehav Rev 18:355-372.

Samson, H. H., Haraguchi, M., Tolliver, G. A. and Sadeghi, K. G., (1989): Antagonism of ethanol-reinforced behavior by the benzodiaepine inverse agonist Ro 15-4513 and FG 7142: relationship to sucrose reinforcement. Pharmacol. Biochem. Behav. 601-608.

Samson H H, Hodge C W (1996): Neurobehavioral regulation of ethanol intake. In Pharmacological effects of ethanol on the nervous system (Deitrich R A and Erwin V G eds) pp 203-226, Press, New York:CRC.

Shannon, H E, Gruzman F, Cook J M (1984): β-carboline-3-carboxylate-t-butyl ester: A selective BZ1 benzodiazepine receptor antagonist. Life Sciences 35:2227-2236.

Skolnick P, Hu R J, Cook C M, Hurt S D, Trometer, J D, Lui R, Huang Q, Cook J M (1997): [³H] RY 80: A high affinity, selective ligand for y-aminobutyric acidA receptors containing alpha-5 subunits. Jn Phannacol Exp Ther 283:488-493.

Spanagel R, Zieglgansberger (1997): Anti-craving compounds for ethanol: New pharmacological tool to study addictive processes. Trends Phannacol Sci 18:54-59.

Stephens D N, Turski L, Hillman M, Turner J D, Schneider H H, Yamaguchi M (1992): What are the differences between abecarnil and conventional benzodiazepine anxiolytics. In Biggio G, Concas A, Costa E (eds), GABAergic Synaptic Transmission, [Molecular, Pharmacological, and Clinical Aspects]. Advances in Biochemical Pharmacology. Raven Press, NY pp 395-405.

Stratford T R, Kelley A E, Simansky K J (1999): Blockade of GABA_{A} receptors in the medial ventral pallidim elicits feeding in satiated rats. Brain Res 825:199-203.

Suzdak P, Glowa J R, Crawley J N, Schwartz R D, Skolnick P, Paul S M (1986): A selective imidazodiazepine antagonist of ethanol in the rat. Science 234:1243-1247.

Ticku M K, Mhatre M, Mehta A K (1992): Modulation of GABAergic transmission by alcohol. In Biggio G, Concas A, Costa E (eds), GABAergic Synaptic Transmission, Molecular, Pharmacological, and Clinical Aspects. Advances in Biochemical Pharmacology. Raven Press, NY, pp 255-267.

Turner J D, Bodewitz G, Thompson C L, Stephenson F A (1993): Immunohistochemical mapping of gamma-aminobutyric acid type-A receptor alpha subunits in rat central nervous system. In: Anxiolytic β-carbolines: from molecular biology to the clinic (D. N. Stephens, ed), pp 29-49 New York: Springer-Verlag.

Volpicelli J R, Alterman A I, Hayashida M, O'brien C P (1992): Naltrexone and the treatment of alcohol dependence. Arch Gen Psychiatry 49: 876-880

Wafford K A, Bain C J, Whiting P J, Kemp J A (1993a): Functional comparison of the role of γ subunits in recombinant human γ-aminobutyric acidA/benzodiazepine receptors. Molec Pharniacol 44:437-442.

Wafford K A, Whiting P J, Kemp J A (1993b): Differences in affinity of benzodiazepine receptor ligands at recombinant γ-aminobutyric acid_{A} receptors subtypes. Molec Pharmacol 43:240-244.

Wisden H, Laurie D J, Monyer H, Seeburg P H (1992): The distribution of 13 GABA_{A} receptor subunit mRNAs in the rat brain: telencephalon, diencephalon, mesencephalon. J Neurosci 12:1040-1062.

Wong G, Skolnick P (1992a): High affinity ligands for “diazepam insensitive” benzodiazepine receptors. Eur J Pharmacol: Molec Pharmacol Sec 225:63-68.

Wong G, Skolnick P (1992b): Ro15-4513 binding to GABA_{A} receptors: Subunit composition determines ligand efficacy. Pharmacol Biochem Behav 42: 107:110.

Zahm D S and Heimer L (1988): Ventral striatopallidal parts of the basal ganglia in the rat: I. Neurochemical compartmentation as reflected by the distributions of neurotensin and substance P immunoreactivity. J Comp Neurol 272: 516-535.

Zhang P, Koehler K F, Zhang P, Cook J M (1995): Development of a comprehensive pharmacophore model for the benzodiazepine receptor. Drug Design and Discovery 12:193-248.

The present invention is not limited to the embodiments specifically described above, but is capable of variation and modification without departure from the scope of the appended claims.

What is claimed is:

1. A method for reducing anxiety in a patient in need thereof, said method comprising the administration of an effective amount of at least one antagonist of α1 containing GABA_{A} receptors to produce an anti-anxiolytic effect in said patient.

2. The method of claim 1, wherein said at least one antagonist of α1 containing GABA_{A} receptors is βCCt.

3. The method of claim 1, wherein said at least one antagonist of α1 containing GABA_{A} receptors is 3-PBC.

4. The method of claim 1, wherein wherein both βCCt and 3-PBC are administered to said patient.

5. The method of claim 1, optionally comprising the administration of naltrexone to said patient.

6. The method of claim 1, wherein about 20 to about 60 mg of said antagonist are administered to said patient.

7. The method of claim 6, wherein 40 mg is administered to said patient.

8. The method of claim 2, wherein said βCCt is administered to said patient three times a week.

9. The method of claim 3, wherein said 3-PBC is administered to said patient 4 times a week.
